(19) United States
(12) Patent Application Publication
(10) Pub. No.: US 2026/0337561 A1
(43) Pub. Date: Oct. 29, 2026

(54) INNATE LYMPHOID CELLS FOR CELL THERAPY AND BIOMARKERS THEREFOR

(71) Applicant: UNIVERSITY HEALTH NETWORK, Toronto (CA)

(72) Inventors: Sarah CROME, Toronto (CA); Sarah COLPITTS, Toronto (CA); Kyle REID, Toronto (CA); Wenhui CUI, Toronto (CA); Sinthuja JEGATHEESWARAN, Toronto (CA)

(21) Appl. No.: 19/487,809

(22) PCT Filed: May 24, 2024
(86) PCT No.: PCT/CA2024/050696

§ 371 Date: Nov. 25, 2025

Related U.S. Application Data
(60) Provisional application No. 63/469,234, filed on May 26, 2023.

(51) Int. Cl. C12N 5/078; A61K 35/17; A61P 37/06; G01N 33/569

(52) U.S. Cl. CPC C12N 5/0634; A61K 35/17; A61P 37/06; G01N 33/56972

(57) ABSTRACT

There is described herein a method of isolating or enriching a population of cells for IL-10 producing group 2 innate lymphoid cells, the method comprising: providing the population of cells which comprises ILC2s; selecting for cells expressing at least one of three markers: CD86, CD49d and retinoic acid related orphan alpha (RORA) and/or 5 deselecting GATA3, including kits for performing the same. There is also provided use of the isolated or enriched population for cell therapy.

### RELATED APPLICATIONS

This application claims priority to U.S. Provisional Patent Application No. 63/469234, filed on May 26, 2023, the entirety of which is hereby incorporated by reference.

### FIELD OF THE INVENTION

The invention relates to innate lymphoid cells (ILCs), and particularly to IL-10 producing ILCs, their use in cell therapy, and biomarkers therefor.

### BACKGROUND OF THE INVENTION

Innate Lymphoid cells (ILCs) are a family of innate lymphocytes with central roles in maintaining tissue and immune homeostasis (1,2). Unlike T cells, ILCs lack antigen-specific receptors and instead rapidly respond to perturbations of cytokines, alarmins, neuropeptides and hormones in the local microenvironment. ILC family includes cytotoxic members (i.e. NK cells), as well as ‘helper’ subsets (ILC1, ILC2s and ILC3s ) that orchestrate immune responses and have important roles in tissue repair. Of note, ILC2s have been shown to recruit and activate regulatory T cells (Tregs) within tissues in inflammatory contexts (3-5) and were shown in mice to protect from Graft-versus-Host disease (GVHD) (6) and ischemia reperfusion injury (7), supporting key roles in promoting immune and tissue homeostasis.

Recent studies identified an ILC2 subpopulation that produces IL-10 (referred to as ILC2₁₀), that can inhibit immune responses in mouse studies of lung inflammation, neuroinflammation and intestinal inflammation, in addition to being expanded in patients that responded to allergy immunotherapy (16-21). Further murine ILC2₁₀ were also shown to prevent rejection of islets in a murine islet transplant model (56) Collectively, these studies support that ILC2₁₀ have important functions in regulating harmful immune responses.

### SUMMARY OF THE INVENTION

In some examples of the present study, we asked whether human ILC2s have applications in cell therapies for aGVHD and inflammatory/immune conditions. We developed methods to expand human ILCs from peripheral blood, and assessed their therapeutic potential using a humanized mouse models of GVHD. ILC2s expanded using our approach express high levels of IL-10, in line with an ILC2₁₀ phenotype, and we further identify markers of expanded IL10⁺ILC2s. Findings herein support applications of ILC2₁₀ in cell-based therapies for GVHD, HSCT, transplantation and inflammatory/immune conditions.

In an aspect, there is provided a method of isolating or enriching a population of cells for IL-10 producing group 2 innate lymphoid cells, the method comprising: providing the population of cells which comprises ILC2s; selecting for cells expressing at least one of three markers: CD86, CD49d and retinoic acid related orphan alpha (RORA) and/or deselecting for cells that express GATA3.

In an aspect, there is provided a population of cells enriched for IL-10 producing group 2 innate lymphoid cells prepared by the method described herein.

In an aspect, there is provided a use of the population of cells described herein, for use in the in the preparation of a medicament for ameliorating, treating or preventing graft-versus-host disease or graft rejection, an inflammatory condition, an autoimmune disorder, allergy, asthma, promoting transplant graft function, and/or preventing or limiting tissue damage in a human subject in need thereof.

In an aspect, there is provided a kit for enriching a population of cells for IL-10 producing group 2 innate lymphoid cells, the kit comprising reagents for detecting cells expressing at least one of four markers, CD86, CD49d, RORA and GATA3 along with instructions for use.

In an aspect, there is provided a method of generating or enriching for a population of cells for IL-10 producing group 2 innate lymphoid cells, the method comprising: providing the population of cells which comprises ILC2s; inducing overexpression of RORA in the population of cells.

### BRIEF DESCRIPTION OF FIGURES

These and other features of the preferred embodiments of the invention will become more apparent in the following detailed description in which reference is made to the appended drawings wherein:

FIG. 1. Expanded ex vivo human ILCs isolated from healthy blood maintain expression of signature cytokines. To explore the role of human ILC2s in GVHD, human ILCs were isolated from peripheral blood using flow cytometry and expanded ex vivo using ILC subset-specific cytokines, as ILC2s are present in very low abundance in peripheral blood. (A) Representative gating strategy for ILC2s and ILC3s. ILC2s sorted as live, lineage⁻ CD94⁻CD16⁻NKG2D⁻CD127⁺CRTh2⁺CCR6⁻ and ILC3s as CD94⁻CD16⁻NKG2D⁻CD127⁺CRTh2⁻CD117⁺CCR6⁺. Lineage antibodies added to remove non-ILCs were CD3 (OKT3), CD3 (UCHT1), CD4, CD8a, CD14, CD15, CD19, CD20, TCRαβ, TCRγδ, CD33, CD34, CD203c, FCεRI, CD79a and CD138. (B) Cell expansion yields of ILC2s and ILC3s at day 20 and and day 3. Representative (C) and average (D) of intracellular cytokine staining for signature cytokines in CD56^{dim} NK cells, ILC2s and ILC3s at day 20 of expansion after stimulation with phorbol 12-myristate 13-acetate (PMA) and ionomycin. (E) Cytometric bead array analysis of secreted cytokines after plating ILCs at a concentration of 2×10⁵ cells/mL for 16-hour stimulation with 100 U/mL of IL-2.

FIG. 2. Cell therapy with human ILC2s suppresses xenogeneic GVHD. To assess whether human ILC2s could limit GVHD, we employed an established xenogeneic model of GVHD involving PBMC transfer into NOD-scid-IL2Rg^{null} mice (NSG). (A) Overview of the xenogeneic GVHD model to assess whether ILC2s would prophylactically limit GVHD. Briefly, NSG mice are irradiated and receive either PBS, human PBMCs (which induce multiorgan tissue pathology), or PBMCs and ILC2s together. Mice are monitored for symptoms of xenogeneic GVHD including weight loss, survival and a composite xenogeneic GVHD score consisting of hunch, skin inflammation and fur loss, activity, pain and percent weight change. Blood is drawn at 7-day intervals for confirmation of human immune cell engraftment and immunophenotyping. At the humane or experimental endpoint, mice are euthanized, and tissues collected for histology or flow cytometry analysis. (B) Representative engraftment human CD3⁺ CD45⁺ T cells (majority of human CD45⁺ cells) at day 14 post injection of human PBMCs. C, D Average engraftment of human CD3⁺ CD45⁺ (C), or CD4⁺ and CD8⁺ T cells (D), in the blood 14 days post injection across 3 independent experiments (n=21 for NSG mice receiving human PBMC and n=13 for nSG mice receiving human PBMC plus allogeneic ILC2 mice from 3 independent experiments). E, F (E) Representative xenogeneic GVHD score (n=3/group in this experiment), and (F) representative weight loss (n=3/group in this experiment) of 3 independent experiments. G, H Average of xenogeneic (G) GVHD score and (H) weight loss of mice at day 20 post injection across three independent experiments (n=23 for PBS treated NSG mice, n=19 for NSG mice receiving human PBMCs alone and n=14 for NSG mice receiving human PBMC plus allogeneic ILC2 respectively across 3 independent experiments). (I) Survival of mice treated with ILC2s across 3 independent experiments (n=23, 19 and 14 for NSG mice receiving PBS, PBMC and PBMC+ILC2 respectively). Experimental endpoint was day 40 for any mice not reaching human endpoint. (J) Average proportion of CD4⁺ and CD8⁺ T cells of total human CD3⁺ T cells in the blood, bone marrow and spleens of NSG mice at endpoint (n=11 and 13 from PBMC and PBMC+ILC 2 respectively from 3 independent experiments).

FIG. 3. Cell therapy with ILC2s inhibits CD4⁺ and CD8 ⁺ T cell proliferation, intestinal infiltration and CXCR3 expression. Effect of ILC2 transfer on T cell phenotypes and proliferation in xenogeneic GVHD model were assessed by flow cytometry. (A) Representative Ki-67 expression by circulating human CD4⁺ T cells from the blood at end point (B) Average Ki-67 expression in blood, bone marrow or spleen at endpoint by human CD4⁺ T cells from 3 independent experiments (n=11 mice per condition from 3 independent experiments). (C) Representative Ki-67 expression by circulating human CD8⁺ T cells. (D) Average Ki-67 expression in the blood, bone marrow or spleen at endpoint on human CD8⁺ T cells from 3 independent experiments (n=11 mice per condition from 3 independent experiments) (E) Representative human CD3 immunohistochemistry of the colon of mice treated with PBMCs or PBMCs plus ILC2s (10× magnification). (F) Average T cell infiltration in the colon of mice treated with PBS, PBMCs or PBMCs+ILC2s. Quantified using CD3 immunohistochemistry and the HALO algorithm and normalized to background in PBS mice.

FIG. 4. Expanded human ILC3s limit xenogeneic GVHD, but do not inhibit in vivo T cell proliferation and intestinal infiltration as observed with ILC2s. To explore whether ILC3s could also limit GVHD, we assessed the effect of cell therapy with ex vivo expanded human ILC3s in our xenogeneic GVHD model. G, H Representative CXCR3 expression on circulating human CD4⁺ (G) or CD8⁺ (H) T cells. Average CXCR3 expression on human CD4⁺ (I) or CD8⁺ (j) T cells in the blood, bone marrow, spleen, colon, and small intestines at experimental endpoint (n=5 for PBMC and n=5 for PBMC+ILC2 conditions respectively from 2 independent experiments).

FIG. 5. Increased circulating ILC2s is associated with decreased CD4⁺ Th1 cells in HSCT recipients protected from GVHD. PBMCs from healthy donors, and HSCT patients with or without GVHD were assessed for ILC proportions and phenotypes using flow cytometry. A, B Representative flow plots (A) and summary graphs (B) of total CD127⁺ helper ILCs in PBMCs as a proportion of live CD45⁺ lineage negative cells. Lineage markers are CD3, CD4, CD8, CD14, CD19, CD20, CD33, CD34, CD123, CD138, CD303, FCεRI and TCRγδ. C, D Representative (C) and average (D) CD117, CRTh2, CXCR3⁺ ILC1s, CRTh2⁺ ILC2s and CRTh2 CD117⁺ ILC3s in PBMCs as a proportion of live CD45⁺ lineage negative cells. (E) Representative CD4⁺ Th1 and CD8⁺ Tc1 cells in HSCT patients with or without aGVHD development. (F) CD4⁺ Th1 and CD8⁺ Tc1 cells across HSCT patients as a proportion of CD4⁺ and CD8⁺ T cells respectively. (G) Correlation of Th1 and Tc1 cells with ILC2s across all HSCT patients. (H) Correlation of Th1 and Tc1 cells with ILC3s across all HSCT patients.

FIG. 6. Ex vivo expanded IL-10 producing ILC2s express CD49d and CD86. (A) UMAP representation of CITE-seq of expanded ILC subsets. ILC subsets indicated were isolated by flow cytometry, expanded and then stained with CITE-seq antibody cocktail. Each population was also labeled with a unique hashtag antibody to facilitate post sequencing identification. (B) Feature plots showing protein level expression of CD16, CD56 and CD117 and RNA level expression of PTGDR2, GATA3, IL17RB, GNLY, IL13, IL5 and IL-10 on expanded ILC subsets. (C) Representative intracellular IL-10 and amphiregulin expression by expanded CD56^{dim} NK cells, ILC2s and ILC3s after PMA/lonomycin stimulation. (D) Average intracellular IL-10 expression assessed by flow cytometry (n=11-13) and secreted IL-10 by cytometric bead array by expanded ILC subsets (n=12, 19 and 20 for CD56^{dim} NK cells, ILC2s and ILC3s respectively). (E) Volcano plot of top differentially expressed transcripts and antibody-derived tags (ADT) between IL-10⁺ and IL-10 expanded human ILC2s. To validate uniquely expressed surface markers of IL-10⁺ILC2s, flow cytometry was performed on additional donors for CD49d CD86. F-I, Representative and average expression of CD49d (F), CD86 (G), as well as CD117 (H), and KLRG1 (I) by IL-10⁺ and IL-10⁻ ILC2s following PMA/ionomycin stimulation (n=7). (J) Average expression of IL-10 following PMA-ionomycin stimulation on ILC2s based on expression of CD49d and CD86 as a representative IL-10 expression, tSNE clustering based on co-expression and average graphs (n=7).

FIG. 7. ILC2s suppress CD4⁺ and CD8⁺ T cell cytokine production via a combination of IL-4 and IL-10. To determine whether cell therapy with ILC2s protective effects were due to direct effects of ILC2s on allogeneic CD4⁺ and CD8⁺ T cells, we performed in vitro co-cultures. Expanded ILC2s were cultured with naïve CD4⁺ and CD8⁺ T cells that were activated with anti-CD3/anti-CD28 beads. After 4 days in culture, intracellular cytokine staining was assessed by flow cytometry. (A) Representative IFN-γ and TNF-α expression by naïve CD4⁺ T cells cultured with or without allogeneic ILC2s at day 4. (B) Average decrease in IFN-g production or IFN-g and TNF-α co-expression represented as log₂fold change compared to CD4⁺ T cells alone (n=17). (C) Representative IFN-γ and TNF-α expression on CD8⁺ T cells co-cultured with allogeneic ILC2s and stimulated with anti-CD3/CD28 beads over 4 days by representative flow cytometry plots. (D) Average decrease in IFN-g and co-expression of IFN-g and TNF-α representative as log₂fold change compared to CD8⁺ T cells alone (n=8). (E) Representative CD4⁺ and CD8⁺ T cell IFN-g expression when cultured with or without ILC2s in the presence of indicated blocking anti-IL-4 and/or anti-IL-10 antibodies. Effects on fold change in IFN-γ expression by CD4⁺ (F) or CD8⁺ (G) T cells after culture with ILC2s with the addition of anti-IL-4 (n=10), anti-IL-10 (n=10) or anti-IL-4 and anti-IL-10 (n=7) blocking antibodies.

FIG. 8. Cell therapy with ILC2s does impede T cell-mediated graft-versus-leukemia effect. (A) Circulating ILC2s from HSCT patients s as a proportion of lineage negative PBMCs. Patients were grouped based on whether they experienced cancer relapse or not. Follow up periods at time of assessment ranged from 30 to 120 days. (B) Overview of humanized GVL model, whereby leukemic cells (MV4-11 cells) are transplanted into NSG mice. PBMCs are administered at D5 following MV4-11 transfer, and engrafted T cells eliminate MV4-11 cells. (C) Representative MV4-11 cells in bone marrow of NSG mice treated with PBS, PBMCs or PBMCs with ILC2s. (D) Average MV4-11 engraftment in NSG mice treated with PBS, PBMCs or PBMCs with ILC2 from two independent experiments (n=7/group) (E) Average xenogeneic GVHD score at day 19 from 2 independent experiments (n=7/group). Day 19 corresponded to 14 days following injection of PBMCs with or without ILC2s.

FIG. 9. General schematic of isolation and expansion of human ILC2s. PBMCs were isolated using a Ficoll density gradient and are enriched for ILCs using a combination of lineage depletion and purification by FACS sorting. ILC2s were sorted as Lin, CD45⁺, NKG2D, CD94, NKp44⁻, CCR6⁻, CD127⁺, and CRTH2⁺. ILC2s were cultured in their activating cytokines for 20 days.

FIG. 10. (A) Bi-directional lentiviral vectors expressing the non-signaling nerve growth factor receptor (NGFR) or GFP marker gene and ILC-associated transcription factors RORA have already been cloned. Lentivirus was produced by transient four-plasmid 48-hour transfection of HEK 293T cells. (B) General scheme for transduction of human canonical ILC2s.

FIG. 11. RORA expression distinguishes ex vivo human ILC2s. UMAP plots of ex vivo ILC subsets after cellular indexing of transcriptomes and epitopes sequencing (CITEseq), and corresponding RNA expression of GATA3 and RORA.

FIG. 12. Ex vivo expanded IL-10 producing ILC2s express RORA, and little to no GATA3, whereas ILC2s that lack IL-10 production express GATA3. (A) UMAP plots of expanded ILC subsets after CITE-seq. ILC subsets were isolated using FACS, expanded and separately stained with an antibody cocktail. Each cell population was stained with a unique hashtag antibody to allow identification post sequencing. (B) UMAP plots showing RNA level expression of IL-10, RORA and GATA3 on expanded ILCsubsets.

FIG. 13. Human ILC2s expanded from healthy blood PBMCs in vitro maintain expression of signature cytokines and produce immunoregulatory cytokine IL-10. Representative flow plots of intracellular cytokine staining in expanded human ILC2s, after stimulation with PMA/lonomycin for 6 hours. Protein inhibitors, Brefeldin A and Monensin were added after 2 hours of stimulation.

FIG. 14. RORA expression positively correlates with IL-10 production by ILC2s. (A) Representative flow plots of transcription factor staining of expanded human ILC2s. (B) Correlation of IL-10 expression versus expanded GATA3⁺ILC2s, and expanded RORA+ILC2s. Flow cytometry expression of IL-10 after ICS of expanded GATA3⁺ and RORA+ILC2s shown as representative flow plots and correlation graphs. Correlation graph of secreted IL-10 versus GATA3⁺ ILC2 and RORA⁺ ILC2s. (C)

Correlation graphs of ILC-associated cytokines versus GATA3⁺ ILC2s and RORA⁺ ILC2s.

FIG. 15. Human canonical ILC2s were transduced with NGFR-pCCL and GFP-pCCL. RORA, at a multiplicity of infection of 2. Transduction efficiency was measured 7 days post-transduction by NGFR or GFP expression.

FIG. 16. Overexpression of RORA induces IL-10 and IL-22 production in ILC2s. (A) Log₂ fold change of IL-10 production calculated using the NGFR-pCCL control transduced ILC2s, and (B) Summary plots of secreted IL-10 measured after CBA by GFP-pCCL. RORA transduced ILC2s showing secreted IL-10 and Log₂ fold change of secreted IL-10 calculated using the NGFR-pCCL control transduced ILC2s.

FIG. 17. Overexpression of RORA upregulates surface expression of CD49d, a marker defining IL-10 producing ILC2s. A) Surface marker expression by GFP-pCCL. RORA ILC2s were quantified by flow cytometry. NGFR-pCCL control transduced ILC2s were tested in parallel to compare changes in surface marker expression (n=1) and B) Flow cytometry expression of CD49d and CD86 expression by GFP-pCCL. RORA transduced ILC2s and flow cytometry expression of CD25, CD117, CD161 and KLRG1 by GFP-pCCL. RORA transduced ILC2s shown as summary plots.

FIG. 18. RORA expression correlates with IL-10 production by ILC2s. A) Representative flow cytometry plots after intracellular cytokine staining (ICS) on expanded ILC2s, showing three high IL-10 producing donors, and three low IL-10 producing donors. Summary plots of IL-10 production across 12 donors. H) Summary plots of production of ILC2 cytokines IL-4, IL-9 and IL-13 by expanded low IL-10 producing versus high IL-10 producing ILC2s. B) Feature plot and violin plot of protein level single cell IL-10 expression on GATA3⁺ and RORA⁺ ILC2s. C) Violin plots of protein level single cell cytokine expression of IL-5, IL-13, IL-4, AREG, IFNG, IL-22 and IL-17A on GATA3⁺ and RORA⁺ ILC2s.

FIG. 19: Human ILC2s prevent immune-mediated destruction of transplanted islets. NSG mice were injected with 150 mg/kg STZ and transplanted with 1500 HLA-A 2⁺ IEQ under the kidney capsule and monitored for blood glucose levels. After 4 weeks, mice were transfused with 1.5×10⁶ HLA-A2-specific CD4 and CD8 CAR-T cells to induce rejection of transplanted islets with or without cell therapy with 1.5×10⁶ allogeneic ILC2s. A) Experimental overview. B) Representative fasted blood glucose levels over time from one independent experiment, and percent normoglycemic of mice across 3 independent experiments.

### DETAILED DESCRIPTION

In the following description, numerous specific details are set forth to provide a thorough understanding of the invention. However, it is understood that the invention may be practiced without these specific details.

The success of allogeneic hematopoietic stem cell transplant (HSCT) as a therapy for hematological malignancies continues to be limited by the complication of graft-versus-host disease (GVHD). Group 2 and group 3 innate lymphoid cells (ILC2s and ILC3s, respectively) have been linked to protection from GVHD, however assessment of their cell therapy potential has been limited by the scarcity of human ILCs in peripheral blood. In this study, we developed methods to isolate and expand large numbers of circulating human ILC2s and assessed their cell therapy potential in a xenogeneic model of acute GVHD (xenoGVHD). Adoptive transfer of ILC2s decreased the severity of xenoGVHD and prolonged NOD-scid IL2Rγ^{null} (NSG) mice survival when given prophylactically or upon GVHD onset. Mechanistically, ILC2 therapy inhibited allogeneic human T cell proliferation and intestinal infiltration, and reduced proportions of CD4⁺ Th1 and CD8⁺ Tc1 cells. In parallel patient studies, increased ILC2s correlated with decreased proportions of CD4⁺ Th1 cells in HSCT recipients and protection from aGVHD, supporting ILC2s antagonize harmful T cell responses in HSCT recipients. CITE-seq analysis revealed high IL-10 expression by ex vivo expanded ILC2s, and that CD49d and CD86 are expressed by expanded IL-10-producing ILC2s. In vitro studies support that ILC2-derived IL-10 and IL-4 mediate suppression of allogeneic CD4⁺ Th1 and CD8⁺ Tc1 cells cytokine production. Importantly, high proportions of ILC2s were not associated with increased rates of cancer relapse in HSCT recipients, and adoptive transfer of ILC2s did not abrogate graft-versus-leukemic effects in a humanized model of HSCT. Collectively these findings support the cell therapy potential of human IL-10⁺ ILC2s for GVHD following allogeneic HSCT, or other transplant, and for inflammatory/immune conditions

In an aspect, there is provided a method of isolating or enriching a population of cells for IL-10 producing group 2 innate lymphoid cells, the method comprising: providing the population of cells which comprises ILC2s; selecting for cells expressing at least one of three markers: CD86, CD49d and retinoic acid related orphan alpha (RORA) and/or deselecting for cells that express GATA3.

In some embodiments, cells are selected that express CD86. In some embodiments, cells are selected that express CD49d. In some embodiments, cells are selected that express RORA. In some embodiments, cells are selected that express both CD86 and CD49d. In some embodiments, cells are selected that express all of CD86, CD49d and RORA.

In some embodiments, the method further comprises deselecting for cells that express GATA3.

In some embodiments, the population of cells is comprised in a sample from a subject. Preferably, the sample is blood, bone marrow, tissue or a lymphoid organ.

In some embodiments, the method is for isolating IL-10 producing group 2 innate lymphoid cells.

In some embodiments, the enriched population of cells is intended for use in the ameliorating, treating or preventing of graft-versus-host disease, transplant rejection, an inflammatory condition, an autoimmune disorder, allergy, asthma, promoting transplant graft function and/or preventing or limiting tissue damage in a human subject in need thereof.

In some embodiments, the method further comprises administering to a subject in need thereof, a therapeutically effective amount of the population of cells enriched for IL-10 producing group 2 innate lymphoid cells, for ameliorating, treating or preventing graft-versus-host disease, transplant rejection, an inflammatory condition, an autoimmune disorder, allergy, asthma, promoting transplant graft function and/or preventing or limiting tissue damage in the subject.

In some embodiments, the subject has undergone or will undergo a cell transplantation procedure. Preferably, the cell transplantation is stem cell transplantation, the latter preferably being hematopoietic stem cell transplantation.

In some embodiments, the subject has undergone or will undergo a bone marrow transplantation procedure.

In some embodiments, the subject has undergone or will undergo a solid organ transplantation procedure.

In some embodiments, the subject has undergone or will undergo a stem cell derived transplantation procedure of tissues, cells or organs.

In an aspect, there is provided a population of cells enriched for IL-10 producing group 2 innate lymphoid cells prepared by the method described herein.

In an aspect, there is provided a use of the population of cells described herein, for use in the in the preparation of a medicament for ameliorating, treating or preventing graft-versus-host disease or graft rejection, an inflammatory condition, an autoimmune disorder, allergy, asthma, promoting transplant graft function, and/or preventing or limiting tissue damage in a human subject in need thereof.

In an aspect, there is provided a kit for enriching a population of cells for IL-10 producing group 2 innate lymphoid cells, the kit comprising reagents for detecting cells expressing at least one of four markers, CD86, CD49d, RORA and GATA3 along with instructions for use.

In some embodiments, the kit comprises reagents for detecting cells expressing CD86. In some embodiments, the kit comprises reagents for detecting cells expressing CD49d. In some embodiments, the kit comprises reagents for detecting cells expressing both CD86 and CD49d. In some embodiments, the kit comprises reagents for detecting cells expressing CD86, CD49d, and RORA. In some embodiments, the kit comprises reagents for detecting cells expressing CD86, CD49d, RORA, and GATA3.

In an aspect, there is provided a method of generating or enriching for a population of cells for IL-10 producing group 2 innate lymphoid cells, the method comprising: providing the population of cells which comprises ILC2s; inducing overexpression of RORA in the population of cells.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The advantages of the present invention are further illustrated by the following examples. The examples and their particular details set forth herein are presented for illustration only and should not be construed as a limitation on the claims of the present invention.

### EXAMPLE 1

### Methods and Materials

### Study Design

The objective of this study was to evaluate the therapeutic potential of in vitro expanded human ILC2s in a humanized mouse model of GVHD and mechanisms by which ILCs suppress T cells. We verified findings in an HSCT patient cohort using flow cytometry on PBMCs isolated from patients who developed GVHD and those who did not.

To explore cell therapy applications of ILC2s, total ILCs were enriched from PBMCs of healthy donors using magnetic separation and isolated using fluorescence activated cell sorting. Surface markers and intracellular cytokine expression on ILCs and T cells was evaluated by flow cytometry. Secreted cytokines were analyzed using cytometric bead array. In vivo effects of ILC 2s on xenogeneic GVHD was performed in 6- to 10-week-old NSG mice which were randomly allocated to receive PBS, PBMCs alone, or PBMCs with ILC2 treatment. Researchers monitoring mice for weight loss and clinical symptoms were blinded. T cell infiltration into tissue was determined by flow cytometry and IHC and evaluated in a blinded manner.. For in vivo graft-versus leukemia experiments, mice were randomly allocated to receive MV4-11 cells or PBS. 5 days later, mice receiving MV4-11 were randomly assigned treatment with PBS, PBMCs or PBMCs with ILC2s. Power analysis was not preformed. Cohort sample size for in vivo studies varied based on availability of expanded ILCs but were performed with at least 3 mice per group in all independent experiments.

CD4⁺ and CD8⁺ T cells for in vitro experiments were isolated from PBMCs using magnetic separation and co-cultured with expanded ILC2s. RNA and surface protein expression on expanded ILC2s, ILC3s, CD56^{dim} NK cells and CD56^{bright} NK cells was evaluated by single cell CITE-seq. Pathways involved in suppression of T cell inflammatory cytokines was determined by addition of neutralizing antibodies. All in vitro assays were performed in duplicate, and each experiment was performed at least 3 independent times.

Sample sizes for patient studies were determined based on availability of samples and acquired freshly or from the Messner Allogeneic Transplant Program Biobank at the University Health Network. Human samples acquired from patients undergoing HSCT were approved the Research Ethics Board at University Health Network in accordance with the Helsinki Declaration (UHN REB 19-6351). Healthy peripheral blood was obtained from donors through the Canadian Blood Services Blood4Research program, with each donor providing written, informed consent (UHN REB 17-6229, CBS Approved Study 2020-047). Animal study protocols were approved by the University Health Network Animal Care Committee (UHN AUP 6203) and all ethical regulations were followed.

### Statistics

Statistical significance was determined by Kruskal-Wallis test or one-tailed Mann-Whitney test. The log-rank (Mantel-Cox) test was used for Kaplan-Meir curves. Analysis of correlational data was calculated using Spearman correlation. The number of replicates is represented by n and is shown in each figure legend. *p<0.05 ;**P<0.01 ;***P<0.001 ;****P<0.0001;ns, not significant. Error bars represent standard deviation unless otherwise stated. Data analysis was preformed using GraphPad Prism v9.

### Human PBMC Isolation

Fresh blood was collected in EDTA collection tubes (BD Biosciences). In some instances, frozen PBMC samples were obtained from the Messner Allogeneic Transplant Program Biobank. PBMCs were isolated using Lymphoprep (StemCell Technologies) per manufacturer instructions.

### Flow Cytometry

Surface marker staining was performed for 30 min after 15 min blocking with human TruStain FcX (BioLegend). Cells were washed in FACS buffer (PBS+2% FCS) and fixed in 2% paraformaldehyde in PBS (ThermoFisherScientific). For intracellular staining, cells were fixed and permeabilized using FOXP3/Transcription Factor Staining set (eBioscience) and incubated with intracellular antibodies at room temperature (RT) for 30 min. Samples were acquired on a LSR Fortessa (BD Biosciences) and data analyzed using FlowJo v10 software.

### Human ILC Sorting

PBMCs were stained with human TruStain FcX (BioLegend) and incubated with a cocktail of lineage antibodies conjugated to the FITC. Cells are washed in FACS buffer, resuspended in EasySep Buffer (StemCell Technologies) and enriched using the EasySep FITC Positive Selection Kit II (StemCell Technologies) per manufacturer instructions. Enriched cells were stained with antibody cocktail, and sorted using a FACSAria Fusion (BD Biosciences).

### Human ILC Expansion

Sorted human ILC2s and ILC3s were cultured in complete X-Vivo 15 media (Lonza) supplemented with 5% human AB serum (Sigma), 100 U/mL Penicillin-Streptomycin (Gibco) and 1x GlutaMAX (Gibco). CD56^{bright} and CD56^{dim} NK cells were cultured in NK MACS media (Miltenyi) supplemented with 5% human AB serum. ILC2s were expanded using 100 IU/mL IL-2(SteriMax), 20 ng/ml IL-7 and 20 ng/mL IL-33 (BioLegend). ILC3s were expanded using 100 IU/mL IL-2, 20 ng/ml of IL- 1β, IL- 7 and IL-23 (BioLegend). CD56^{bright} and CD56^{dim} NK cells were expanded using 500 IU/mL of IL-2, and 20 ng/ml of IL-15 and IL-18 (Biolegend).

### Cytokine and Chemokine Assays

For intracellular cytokine analysis, 2×10⁵ cells were stimulated with Cell Stimulation Cocktail (eBioscience) for 6 hrs, with GolgiStop and GolgiPlug (BD Biosciences) added after 2 hrs. Cells were then intracellularly stained with antibodies. For secreted factor analysis, 1×10⁵ cells were plated in complete X-Vivo media and stimulated with 100 IU/mL of IL-2. After 16 hrs, supernatant was collected and stored at −80 °C. Analytes were measured using the 12-plex LegendPlex Human Th Cytokine Panel (BioLegend) or the 13-plex LegendPlex Human Proinflammatory Chemokine Panel 1 (BioLegend) per manufacturer's instructions.

### Xenogeneic GVHD Mouse Model

Six-to-ten-week-old NOD. Cg-Prkdc^{scid}II2rg^{tm1Wjl}/SzJ (NSG) mice were given 150 cGy irradiation one day prior to intravenous tail vein injection of 1×10⁷ freshly isolated PBMCs with or without 1×10⁷ expanded ILC2s in PBS at the same time or 9 days later (23, 44). Control mice were injected with PBS. Mice were monitored daily for symptoms of GVHD including weight loss, fur loss and skin inflammation, hunch, activity, and pain, scored on a scale of 0-3. At endpoint, blood, bone marrow and spleens were harvested as previously described (44, 45). For intestinal immune cells, intestines were flushed, sliced, and rinsed in 1xPBS. Tissue was cut into segments, added to 2 mM EDTA in 1xPBS and incubated for 30 min at RT. Tissue was strained and the remaining tissue was added to digestion buffer containing 1,650NPA-U BP protease (VitaCyte), 2,500CDA-U CollagenaseMA (VitaCyte), and 100 μg/mL DNasel (StemCell Technologies) in Hank's Balanced Salt Solution with calcium (Wisent Bio Products). Tissue was then cut and incubated at 37 °C for an additional 30 min. Tissue was passed through a 70 μm filter and washed in RPMI (Gibco)+5% FCS twice.

For graft-versus leukemia experiments, six-to-ten-week-old NSG mice were given 250 cGy irradiation one day prior to intravenous tail vein injection of 2×10⁶ MV 4-11 cells per mouse. Five days later, mice were given 5×10⁶ PBMCs alone or with 5×10⁶ expanded human ILC2s. Mice were sacrificed 14 days after PBMC injection and bone marrow was harvested.

### Histology

Harvested spleen, lung and colon tissue were fixed for 3 days in 10% neutral buffered formalin and stored in 70% ethanol. Paraffin embedding, tissue slicing, H&E and immunohistochemical staining was performed by the UHN Pathology Research Program Laboratory. Quantification of cells expressing IHC markers was performed using the HALO Image Analysis Platform and reported as a percent of all cells.

### Co-Cultures

Naïve CD4⁺ and total CD8⁺ T cells were isolated from the healthy donor blood using the EasySep Human Naïve CD4⁺ T cell or the EasySep Human CD8⁺ T cell isolation kit (StemCell Technologies). T cells were plated at 5×10⁴ per well and stimulated with human CD3/CD28 T-Activator DynaBeads (Gibco) at 1:8 beads: T cell ratio. Expanded ILC2s were added 1:1 to the T cells. T cells were assessed for markers after 72 hrs by flow cytometry. For ILC supernatant experiments, expanded ILC2s were plated 5×10⁴ cells/mL in complete X-Vivo media without cytokines. After 16 hrs, supernatant was harvested, centrifuged at 1500 rpm for 10 min and stored at −80 °C. Supernatant was thawed on day of T cell cultures, and added 1:1 with T cells. Fresh supernatant was added every 24 hrs for 3 days. For inhibitor experiments, UltraLeaf anti-human IL-4, IL-9, IL-10, and IL-13(BioLegend) were added at 10 g/mL. ARL67156 (Tocris) and PBS12379 (Tocris) were added at 1 μg/mL to inhibit activity of CD39 and CD73, respectively. Inhibitors were added at day zero and every 24 hrs for 72 hrs.

### Single-Cell Cite Sequencing

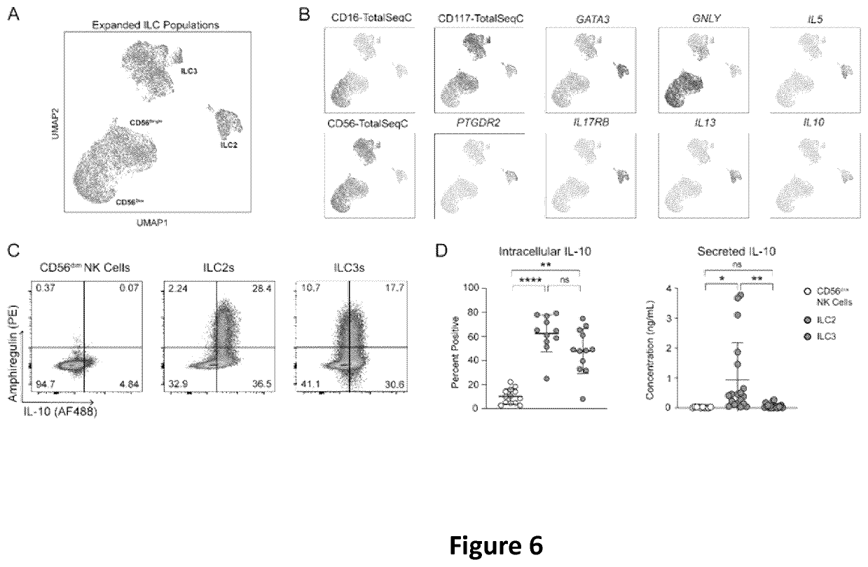

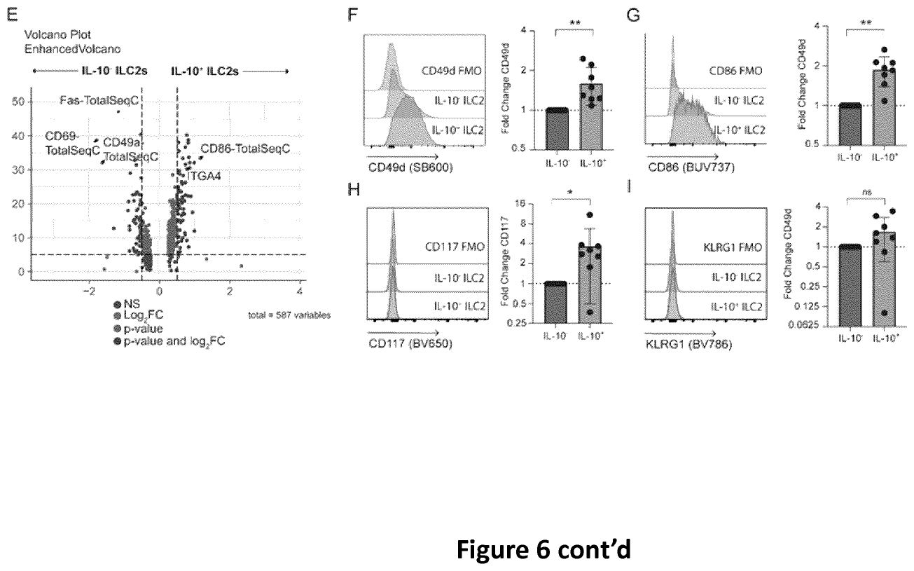

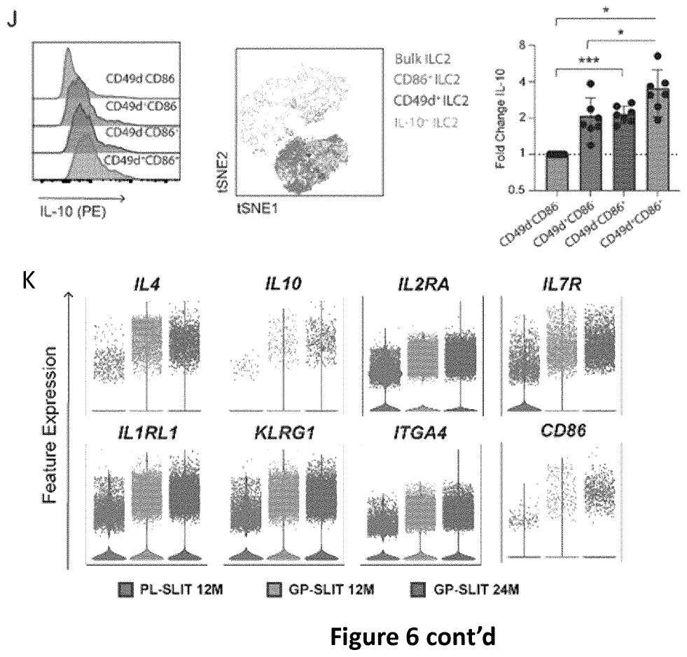

1×10⁶ expanded ILCs were washed twice in 1x Cell Staining Buffer (BioLegend) and incubated in TruStain FcX for 15 min. Cells were incubated with a TotalSeq-C antibody cocktail (BioLegend) for 30 min. Cells were washed 3x in Cell Staining Buffer and resuspended in 1xPBS with 0.04% BSA (Millipore Sigma). Samples were prepared for sequencing using the 10X Genomics Single Cell 5′ v2 platform in accordance with manufacturer's instructions for capture of 12,000 cells per samples. Reverse transcription, cDNA amplification and sequencing libraries using the 10X Genomics Single Cell 5′ v2 reagents. Samples were sequenced to a depth of 40,000 reads. Read alignment to the reference human genome (GRCh38/hg38) and gene expression matrices were generated using CellRanger v6.1.2. Single cells were filtered to exclude cells that expressed >10% mitochondrial content, <1000 total transcripts and <200 unique genes. Data was log normalized, principal component analysis was preformed, and cells were clustered by the top 20 principal components with the Louvain community algorithm using Seurat's FindNeighbors and FindClusters (46). Clusters were visualized using the Uniform Manifold Approximation and Projection (UMAP) (47). Clusters were assigned to ILC subsets based on expression of hashtag antibodies, lack of lineage markers and expression of subset associated markers (FIG. 6a). Cluster defining markers were identified using FindMarkers in Seurat. Differential expression of genes was visualized using EnhancedVolcano (48)

### Data Availability

CITEseq dataset will be made publicly available via NCBI GEO at the time of publication

### Study Approval

Patient study protocols were approved by the Research Ethics Board at University Health Network in accordance with the Helsinki Declaration (UHN REB 19-6351), with all patients provided written, informed consent. Healthy peripheral blood was obtained from donors through the Canadian Blood Services Blood4Research program, with each donor providing written, informed consent (UHN REB 17-6229, CBS Approved Study 2020-047). Animal study protocols were approved by the University Health Network Animal Care Committee (UHN AUP 6203) and all ethical regulations were followed.

### Results and Discussion

### Human ILC2s and ILC3s Can Be Expanded Without Alteration of Cytokine Profile

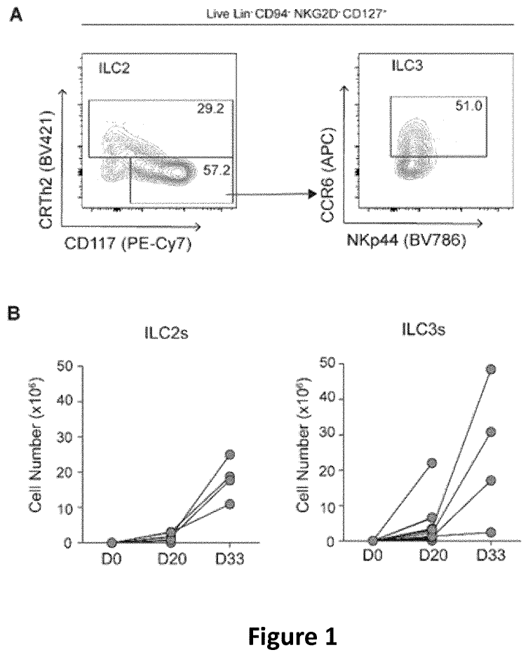

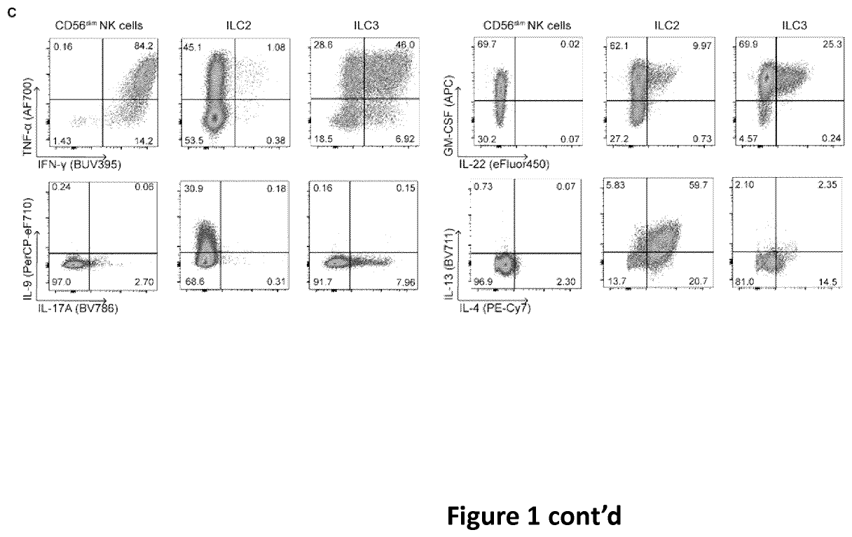

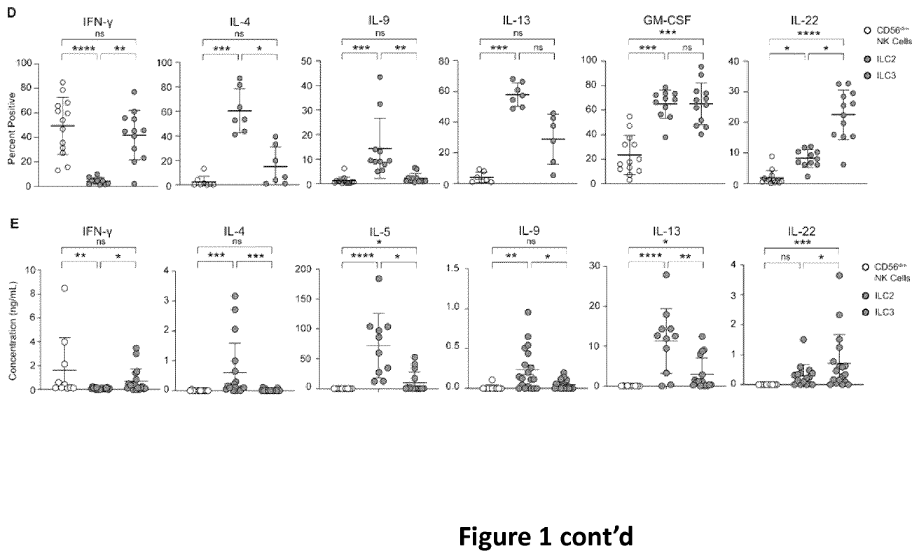

To examine ILC2s and ILC3s cell therapy potential, we first had to develop methods to isolate and expand ILC2s from peripheral blood in a manner that maintains signature cytokine expression, as they are present in very low abundance. We isolated ILCs from normal donor peripheral blood mononuclear cells (PBMCs) by flow cytometry sorting using a combination of ILC markers and gating out lineage positive cells (FIG. 1A). CD56^{dim} NK cells were sorted as live lineage CD45⁺CD56⁺CD16⁺, ILC2s as live lineage⁻CD45⁺CD94⁻CD16⁻NKG2D⁻CD127⁺CRTh2⁺CCR6⁻ and ILC3s as live lineage ⁻CD45⁺CD94⁻CD16⁻NKG2D⁻CD127⁺CRTh2⁻CD117⁺CCR6⁺. NK cells were expanded in NK MACS media supplemented with IL-2, IL-15 and IL-18, ILC2s were then expanded in X-Vivo media supplemented with IL-2, IL-7 and IL-33, and ILC3s were expanded in X-Vivo media with IL-1β, IL-2, IL-7 and IL-23. ILC2s and ILC3s exhibited robust expansion using this protocol, resulting in an average of 2.3×10⁴±7.2×10³ and 9.8×10³±3.6×10³ fold expansion respectively after 34 days in culture (FIG. 1B). Post-expansion, ILC2s strongly co-expressed IL-4, IL-13, IL-9 and GM-CSF following PMA-ionomycin stimulation, with low to no expression of IFN-γ, IL-17A or IL-22 (FIG. 1C, D). In contrast, expanded CD56^{dim} NK cells co-expressed IFN-γ and TNF-α and ILC3s expressed IL-22 and GM-CSF, with low IL-17A expression (FIG. 1C, D). Secreted cytokine production was quantified using cytometric bead array (CBA) following stimulation of ILC subsets with IL-2. ILC2s secreted high amounts of IL-4, IL-9, IL-5 and IL-13, whereas NK cells and ILC3s did not produce significant amounts of these cytokines and instead produced canonical cytokines including IFN-γ and TNF-α or IL-22, respectively (FIG. 1E). Expanded ILC2s also produced the chemokines CCL2, CCL11, CCL20 and CXCL10. Further, expanded ILC2s maintained cytokine expression when cultured in ILC3 or NK cell medium and ILC3s maintained an ILC3-associated cytokine profile when cultured in ILC2 or NK cell-associated cytokines, supporting both ILC populations exhibit a stable phenotype.

### Adoptive Transfer of Allogeneic Human ILC2s Suppresses Xenogeneic GVHD

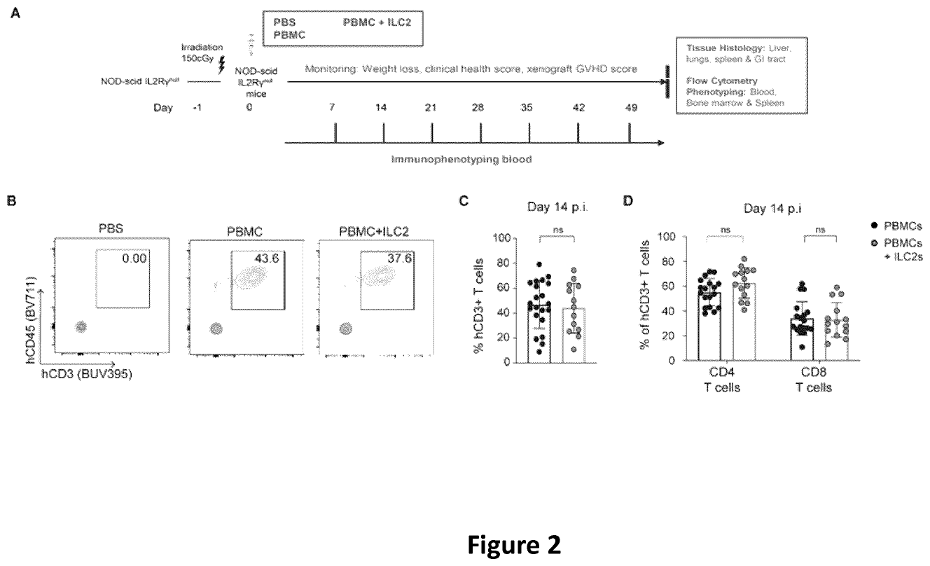

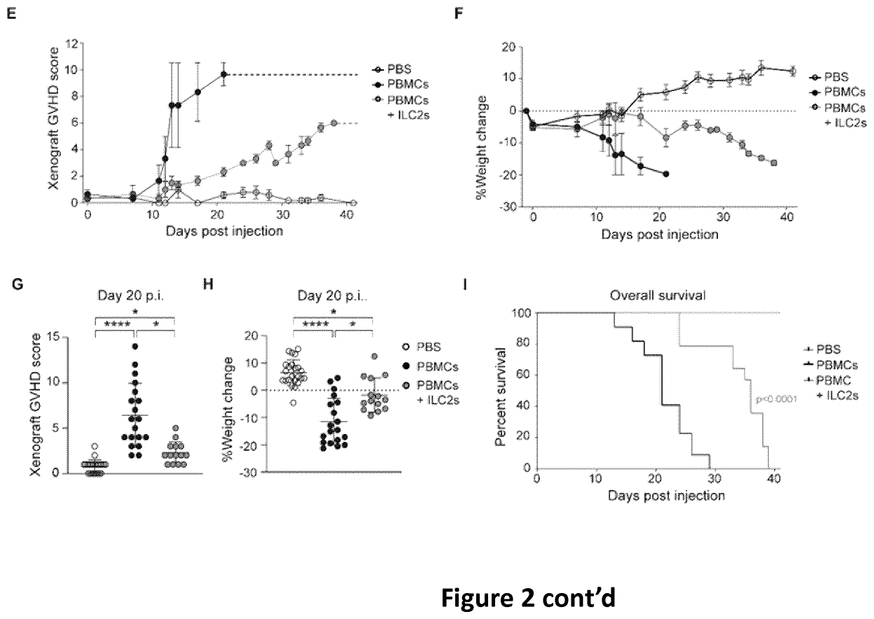

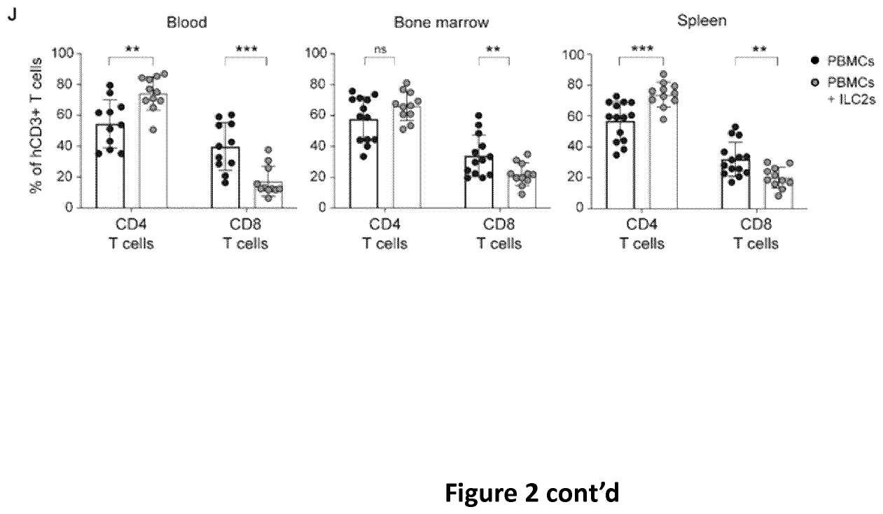

Due to prior mouse studies supporting potential applications of ILC2 transfer for GVHD (15, 22), we first assessed human ILC2s cell therapy potential using a xenogeneic GVHD model (xenoGVHD). 6-8-week-old female NOD-scid-IL 2Rg^{null} mice were irradiated and injected with human PBMCs to induce tissue pathology similar to aGVHD after HSCT (23). NSG mice were monitored for signs of xenoGVHD using weight-loss and a composite xenoGVHD score (FIG. 2A). After 14 days, engraftment of human CD45⁺CD3⁺ T cells was observed in the blood, with no significant difference in T cell proportions between NSG mice given human PBMCs alone or mice treated with ILC2s, indicating ILC2s did not inhibit T cell engraftment (FIG. 2B-D).

NSG mice were monitored for signs of xenogeneic GVHD (xenoGVHD) including weight-loss and a composite xenogeneic GVHD score, which measures fur loss, skin inflammation, hunch, activity, and pain. Across 3 independent experiments, NSG mice treated with ILC2s had significantly delayed onset and reduced GVHD symptoms, including decreased weight-loss (FIG. 2E-H). These effects were also observed in parallel experiments in male NSG mice, although with different kinetics. This single infusion of ILC2s not only reduced xenoGVHD severity but also significantly improved survival in this model. Notably ILC2-treated mice only reached endpoint due to weight-loss but other GVHD symptoms were still low (FIG. 21) and would otherwise not have reached endpoint. To assess if ILC2s could also treat GVHD upon onset, allogeneic ILC2s were adoptively transferred after T cell engraftment and onset of xenoGVHD symptom, resulting in a similar protection from xenoGVHD than when ILC2s were administered prophylactically at day 0. Taken together, cell therapy with human ILC2s limited pathology and enhanced overall survival in this model of GVHD.

Allogeneic ILC2s Inhibit T Cells Responses in xenoGVHD Model

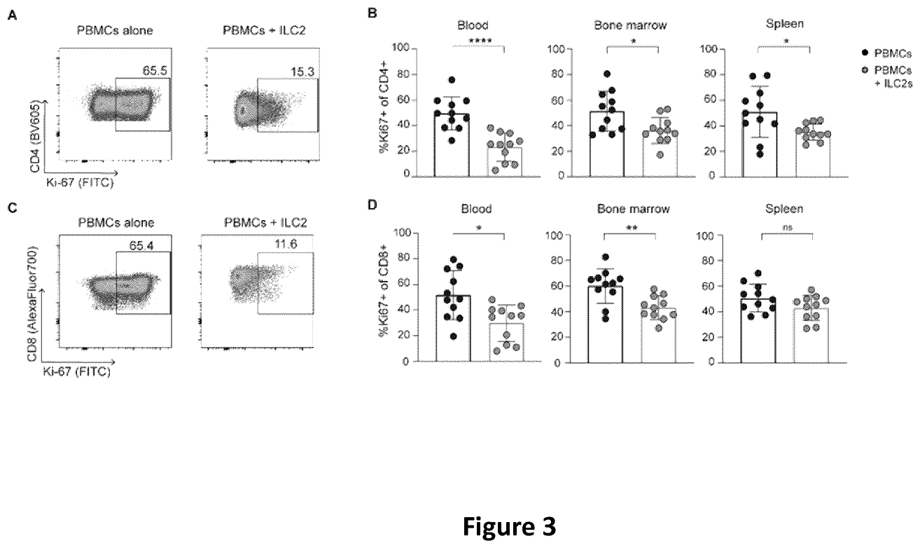

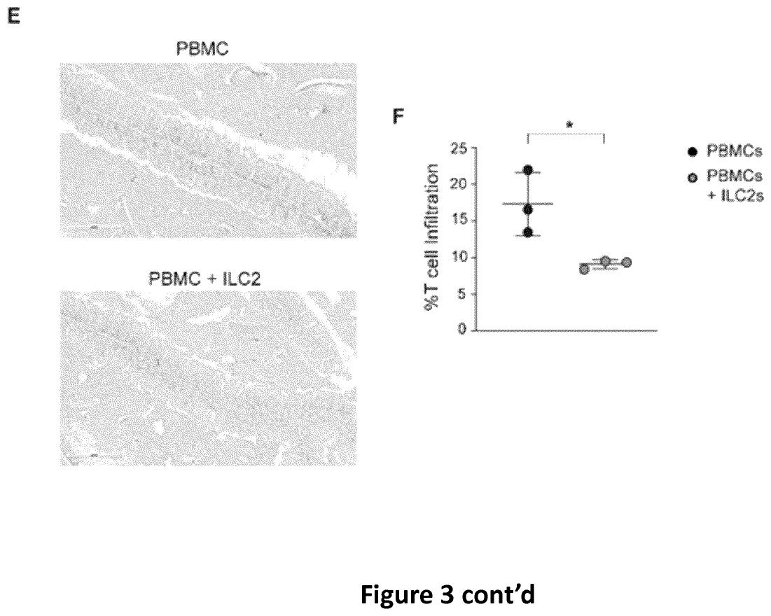

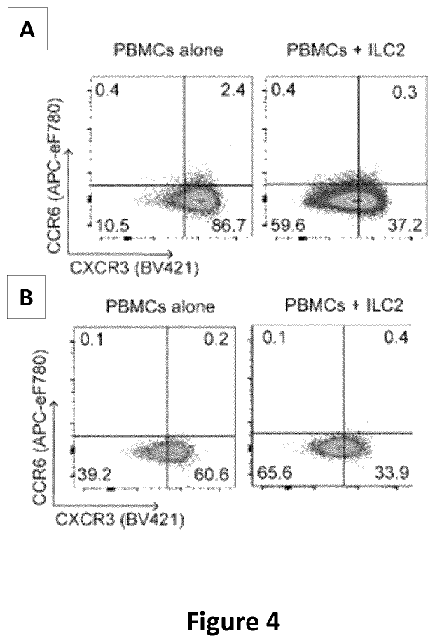

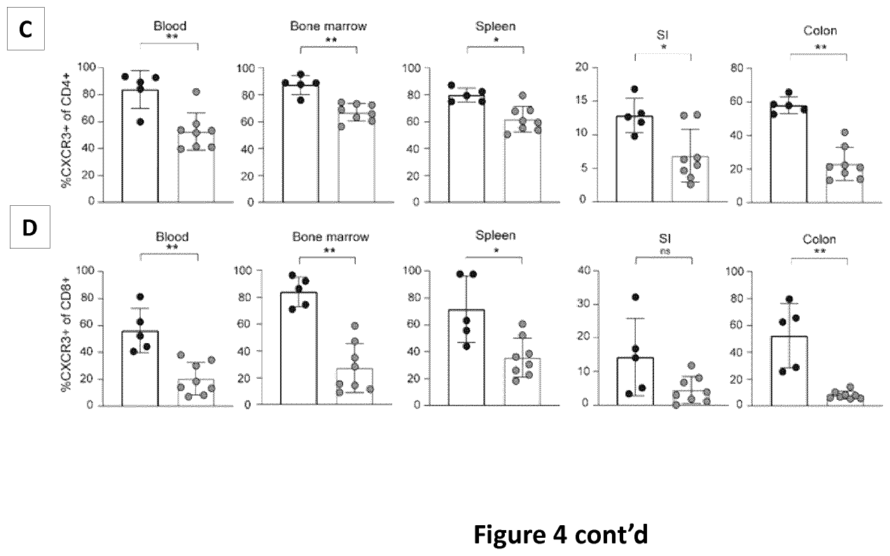

We next sought to determine how ILC2s protected from GVHD. Similar to previous reports, T cells made up >96% of engrafted human CD45⁺cells across all organs analyzed (FIG. 2B-D) (23). Despite no differences in engraftment at day 14, we noted at endpoint a moderate increase in CD4⁺ T cells and reciprocal decrease in CD8⁺T cells in the blood, bone marrow and spleen of NSG mice receiving ILC2s (FIG. 2J), indicating T cell responses were altered following ILC2 therapy. We observed that blood, bone marrow and splenic CD4⁺ and CD8⁺ T cells had reduced expression of Ki-67 in NSG mice treated with ILC2s, indicating reduced proliferation of T cells (FIG. 3A-D). This was not accompanied by changes in T cell expression of checkpoint molecules including PD-1, CTLA-4 and CD25.

Previous research has indicated that CXCR3⁺ T cells in mouse models of GVHD drives trafficking to target organs, including intestines (24, 25). CXCR3 also marks CD4 ⁺ Th 1 and CD8⁺ Tc1 cells (26-28) which induce intestinal damage in GVHD models and correlate with disease in patients (2, 4). We therefore asked whether ILC2s influenced T cell phenotypes and intestinal trafficking within the xenoGVHD model. CD3 immunohistochemical (IHC) staining of tissues revealed a stark reduction in T cell infiltration into colons of ILC2-treated mice, with no change in T cell infiltration noted in the lungs, spleen, or liver (FIG. 3E, F). Across compartments examined, mice receiving ILC2 cell therapy had reduced CXCR3 expression by CD4⁺ and CD8⁺ T cells (FIG. 3A-D), with no increase in CRTh2 or CCR6, markers of CD4⁺ Th2 and Th17 cells, respectively (27-29). Adoptive transfer of ILC2s therefore did not promote differentiation to other T cell subsets, but inhibited CD4⁺ Th1 and CD8⁺ Tc1 cells (FIG. 3A-D). (27-29).

### HSCT Recipients Without GVHD Have Elevated Proportions of ILC2s

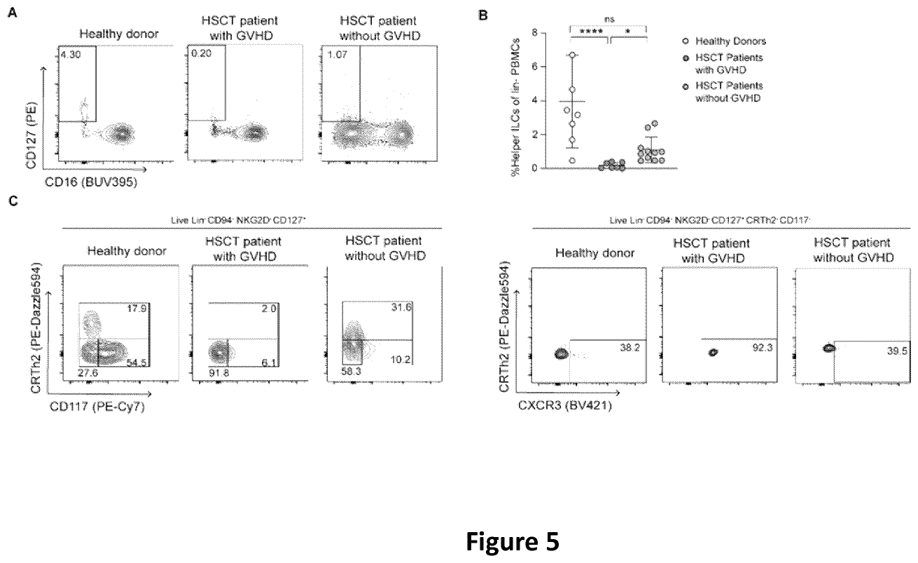

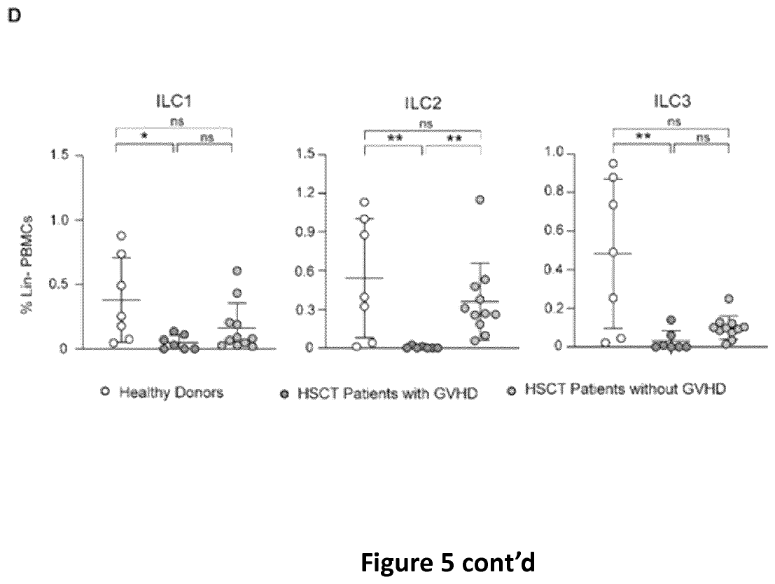

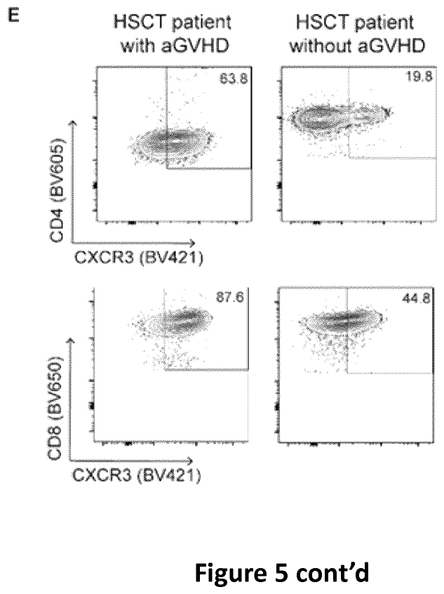

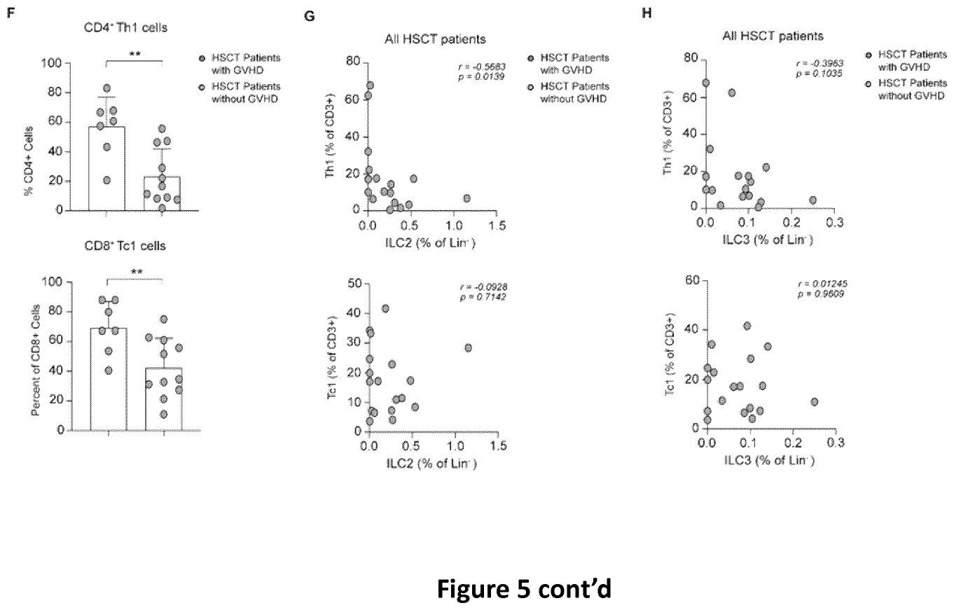

Prior studies have reported increased numbers of CD56^{bright} NK cells, ILC2s and ILC3s in peripheral blood of HSCT patients that do not develop GVHD compared to those that do (12). Due to ILC2-mediated suppression of CD4⁺ Th1 and CD8⁺ Tc1 cells in the xenoGVHD model, we were interested in the relationships between ILCs and T cell subsets in HSCT recipients with differing clinical outcomes. We analyzed the proportion of subsets of ILCs and T cells in the blood of HSCT patients at the time of aGVHD diagnosis and compared proportions of these populations in patients without aGVHD and healthy donors. All HSCT recipients were on prophylactic post-transplant cyclophosphamide, and two were treated with additional immunosuppression at the time of sample acquisition. In line with previous literature, patients without aGVHD had decreased proportions of CD56^{dim} NK cells and increased proportions of CD56^{bright} NK cells (10, 11). Similarly, patients that developed aGVHD had a reduced proportion of helper ILCs (Lin⁻ CD127⁺) (FIG. 5A, B), with reduced proportions in ILC1s, ILC2s and ILC3s compared to healthy controls (FIG. 5C, D). HSCT patients without aGVHD, however, had significantly higher proportions of ILC2s, but differences in ILC1s or ILC3s compared to patients with aGVHD with our sample size was not significant (FIG. 5C, D).

We next examined the correlation of ILCs with T cell subsets in HSCT recipients. Similar to what we observed with ILC2 therapy in the xenoGVHD model, HSCT recipients with aGVHD had increased CD4⁺ Th1 and CD8⁺ Tc1 cells as a proportion of total CD4⁺ and CD8⁺ T cells respectively (FIG. 5E, F), with no significant difference in Th17 cells (26-28). Elevated ILC2s cells strongly correlated with low proportions of Th1 cells as a percent of CD3⁺ T across all HSCT patients (FIG. 5G). The proportion of ILC3s, however, was not significantly correlated with changes in Th1 or Tc1 cells across HSCT patients (FIG. 5H). Taken with our findings that ILC2 therapy suppresses xenoGVHD and inhibits allogeneic CD4⁺ Th1 and CD8⁺ Tc1 cells, normal human ILC2s proportions following HSCT protect from aGVHD and are associated with decreased pathogenic T cell responses.

### Expanded ILC2s Display ILC2¹⁰ Phenotype

To identify potential mechanisms by which ILC2s could inhibit allogeneic T cells, we preformed single cell Cellular Indexing of Transcriptomes and Epitopes by Sequencing (CITE-seq) on expanded ILCs. Expanded ILCs were stained with a TotalSeq-C antibody cocktail and labelled with hashtag antibodies to enable easy identification after sequencing (FIG. 6A). All ILCs examined expressed lineage defining markers that differentiated them from other family members. CD56^{bright} and CD56^{dim} NK cells expressed GNLY and CD56, while the CD56^{dim} NK cells expressed CD16. ILC3s strongly expressed CD117 and ILC2s expressed PTGDR2, GATA3, IL17RB, IL13 and IL5 (FIG. 6B).

We assessed expression of molecules linked to regulatory CD4⁺FOXP3⁺ Tregs and T regulatory 1 (Tr1) cells. Low or background expression of checkpoint molecules such as PD-1, PD-L1, CTLA-4 or LAG3, was observed at both the RNA and protein level, as was the FOXP3 gene (30, 31). Additionally, we did not detect IL12A and EBI3, which make up the regulatory cytokine IL-35. Despite ubiquitous transcriptional expression of TFGB1 across ILC subsets, there was no protein-level expression of active TGF-β1 in expanded NK cells, ILC2s or ILC3s. While these regulatory molecules were absent, both expanded ILC2s and ILC3s expressed ENTPD1, the gene for CD39. ILC2s and ILC3s also weakly expressed NT5E (CD73), however only ILC2s and CD56^{bright} NK cells exhibited surface CD73 protein expression. Flow cytometry analysis verified expression of these enzymes on expanded ILCs, with ILC2s co-expressing high levels of CD39 and CD73, which are known to inhibit the functional activity of T cells (32, 33). Intriguingly, one of the top genes identified as being uniquely expressed by ILC2s was IL-10 (FIG. 6B), which we validated by flow cytometry and cytometric bead array (FIG. 6C, D), where expanded ILC2 supernatants and observed consistently high levels of IL-10 secretion across all donors and noted a high proportion of IL-10⁺ cells in the majority of donors examined (FIG. 6C, D). Therefore ILC2s expanded using our approach were consistently IL-10⁺ILC2s. This was of note, as several recent studies identified IL-10⁺ILC2s as having immunoregulatory properties, including being able to suppress both autologous and allogeneic T cells (17, 21, 34).

Despite consistently high levels of IL-10, we noted heterogeneity in the proportions of IL-10⁺cells between ILC2 isolations from different donors. We asked whether markers of IL-10⁺ILC2s in our expanded ILC2 culture could be identified that could better identify IL-10⁺ILC2s. Differential gene expression analysis was performed comparing the IL10⁺ILC2s to IL10⁻ILC2s to uncover markers that could be used to distinguish IL-10⁺ from IL10⁻ILC2s. Elevated expression of CD86 and ITGA4 (CD49d) was noted on the IL10⁺ILC2s (FIG. 6E). Flow cytometry confirmed high CD49d and CD86 expression by on IL-10⁺ILC2s relative to IL-10⁻ILC2s (FIG. 6.F, G). We also examined CD117 expression, as ex vivo ILC2s had variable expression of CD117 during isolation, and KLRG1 which was previously reported to be expressed by IL-10-producing ILC2(21). Both IL-10⁺ and IL-10⁻ILC2s had very low expression of these markers, although we noted a small but significant fold increase in CD117 expression (FIG. 6.H, I). Dividing expanded ILC2s into populations based on expression of CD49d and/or CD86 revealed co-expression of CD49d and CD86 had the highest IL-10 expression relative to CD86-CD49d ILC2s (FIG. 6.J). CD49d and CD86 expression therefore marks ex vivo expanded IL-10⁺ILC2s, which could be used to increase purity of IL-10⁺ILC2s in expansion protocols.

To assess the potential utility of these markers to identify ILC2₁₀ in patient-based studies, we examined the publicly available scRNA-seq dataset of ILCs from patients with grass-pollen allergy treated with either grass-pollen sublingual allergen-specific immunotherapy (GP-SLIT) or placebo (PL-SLIT) (21). Here, the authors reported that patients treated with GP-SLIT had improved outcomes and increased proportions of ILC2₁₀ at 12-month and 24-month follow-ups compared to placebo treated patients. Further, the authors reported KLRG1 expression defined ILC2₁₀, and pathways related to IL-10 signalling and regulation were upregulated in at both time points following GP-SLIT. We re-analyzed this scRNA-seq dataset to evaluate whether the increased IL-10 expression by ILC2s was associated with increases in CD49d and CD86. Following clustering of ILC2s from GP-SLIT treated patients, there was increased expression of IL10 as reported, as well as IL4 (FIG. 6K). Further, GP-SLIT treated patients had elevated expression of IL2RA, IL7R and IL1RL1, receptors for the cytokines used for our ILC2₁₀ expansion (FIG. 6K). Notably, ILC2s from GP-SLIT treated patients had elevated expression of ITGA4 (CD49d) and CD86, in addition to elevated KLRG1 expression as reported (FIG. 6K). This elevated expression of CD49d and CD86 that correlated with ILC2₁₀ supports the potential broad applications of these markers to differentiate ILC2₁₀ from inflammatory ILC2s in human studies.

Expanded ILC2s Inhibit CD4⁺ and CD8⁺ T Cells via IL-4 and IL-10

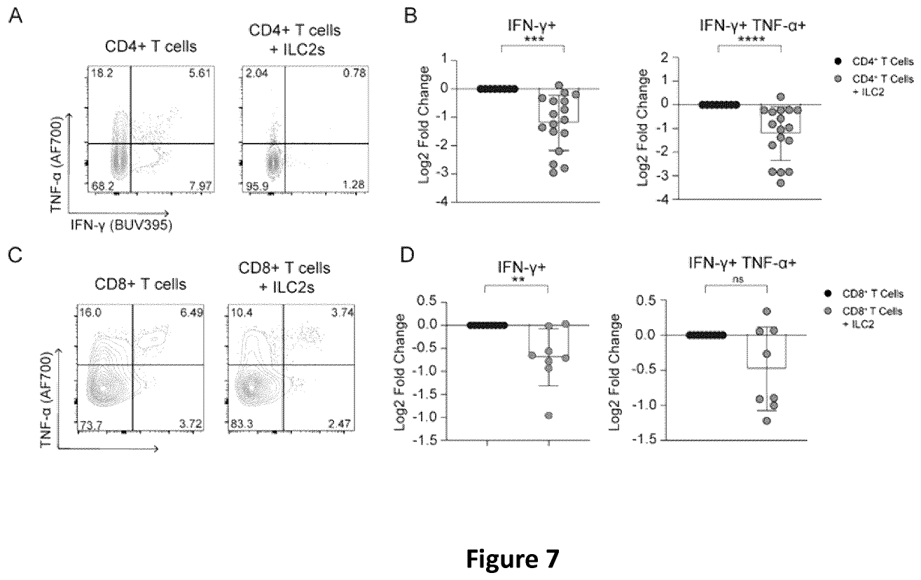

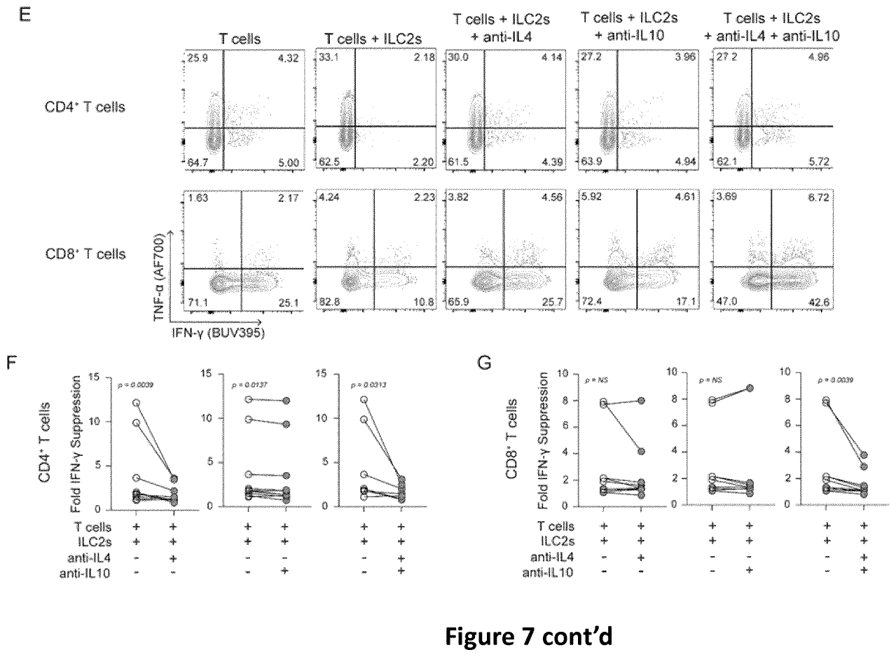

To determine whether ILC2s or ILC3s could directly regulate allogeneic T cells, in vitro co-cultures were performed with bulk ILC2s or ILC3s and allogeneic naïve CD4⁺ or CD8⁺ T cells. Culture with ILC2s resulted in a dramatic reduction in IFN-γ⁺ and IFN-γ⁺/TNF-α⁺ T cells (FIG. 7A, B). Decreased IFN-γ was also observed when allogeneic ILC2s were cultured with CD8⁺ T cells (FIG. 7C, D). To confirm in vivo findings that ILC2s reduced Th1 and Tc1 cells without increasing proportions of Th2 or Th17 cells, we also examined cytokines expressed by other T cell subsets. No increase in IL-17A, IL-22, IL-4, IL-9 or IL-13 was observed with addition of ILC2s to cultures with CD4⁺ or CD8⁺ T cells. Thus, decreased IFN-γ does not appear to be due to altered T cell differentiation, but ILC2-mediated inhibition of CD4⁺ Th1 and CD8⁺ Tc1 cells. In contrast to ILC2s, ILC3s co-cultured with CD4⁺ and CD8⁺ T cells did not affect cytokine expression, further validating that ILC3s regulate via different mechanisms from ILC2s. This data taken with xenogeneic GVHD data supports that while ILC3s may be protective in GVHD, their functions are distinct from that of ILC2s.

We next explored whether various molecules associated with ILC2s or identified by CITE-seq controlled ILC2-mediated regulation of T cells interactions. We noted that allogeneic T cells activated in the presence of supernatants from IL-2-stimulated ILC2s had comparable suppression of T cells to that of adding ILC2s to T cell cultures directly, indicating one or more secreted factors produced by ILC2s underlies their ability to inhibit T cells.

ILC2s were next cultured with allogeneic T cells in the presence or absence of neutralizing antibodies or inhibitors to molecules of interest (IL-10, CD39, CD73), as well as ILC2 cytokines (IL-4, IL-9, IL-13) (35-37). The addition of an IL-4-neutralizing antibody strongly reduced ILC2-mediated suppression of IFN-γ by CD4⁺ T cells in vitro, but blocking IL-9, IL-13 or CD39/CD73 had no effect (FIG. 7E, F). Neutralizing IL-10 also decreased suppression of CD4⁺ T cell-IFN-γ, but to a lesser extent than IL-4 (FIG. 7E, F). In contrast, the combination of anti-IL-4 and anti-IL-10 abrogated ILC2-mediated suppression of IFN-γ by CD8⁺ T cells, which was not observed with anti-IL-4 or anti-IL-10 alone (FIG. 7E, G). Thus, IL-4 and IL-10 are at least one mechanism whereby ILC2s can suppress allogeneic CD4⁺ and CD8⁺ T cells, with differing contributions of IL-4 and IL-10 to the regulation of CD8⁺ and CD4⁺ T cells.

Allogeneic Human ILC2s Do Not Suppress T Cell Mediated Graft-versus-leukemia Effects

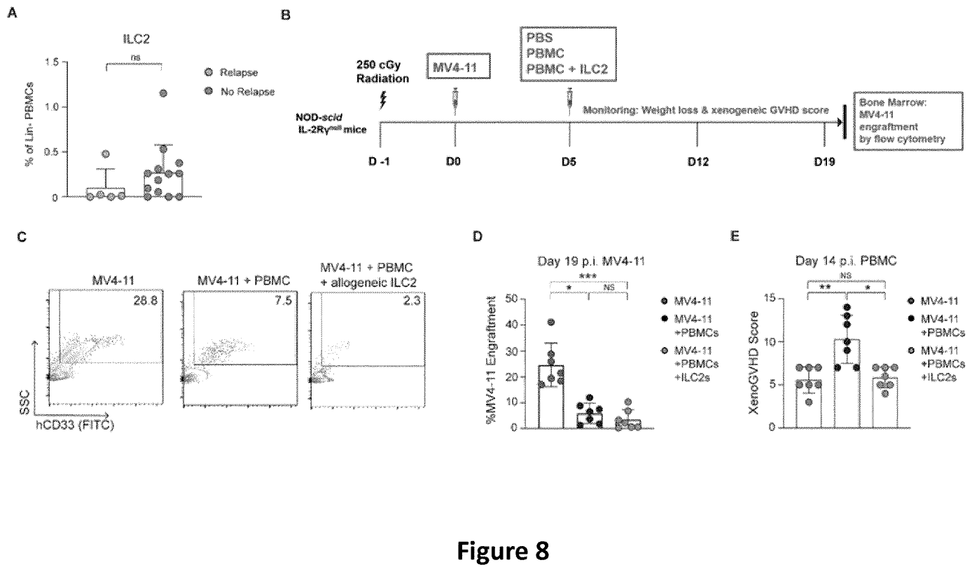

The graft-versus leukemic (GVL) effect is critically important for HSCT effectiveness. As we had observed direct regulation of T cell responses by ILC2 in the xenogeneic GVHD model, and increased ILC2 proportions in patients protected from GVHD development, a key question was whether ILC2s would have detrimental effects T cell-mediated GVL effects. Within our clinical cohort, we assessed circulating ILC2 proportions and how they correlated with relapse status. No significant differences in ILC2s proportions was observed in the blood of patients which went on to experience relapse of their malignancy with our sample size, suggesting that the increased proportion of ILC2s were not inhibiting the GVL effects (FIG. 8A).

We next assessed whether ILC2 cell therapy would negatively impact T cell-mediated GVL-effects in a in a humanized mouse model. Briefly, MV4-11 cells, an acute myeloid leukemia cell line, were injected into mice 5 days prior to transfer of PBMCs alone or PBMCs with expanded allogeneic ILC2s (FIG. 8B). Ability of T cells to kill MV4-11 cells was then assessed at 14 day. As expected, PBMC injection resulted in a reduction in MV4-11 cells in the bone marrow compared to mice receiving MV4-11 cells alone (FIG. 8C, D). Mice treated with PBMCs and allogeneic ILC2s had comparable reductions in MV4-11 cells within the bone marrow to that observed with mice receiving PBMCs alone (FIG. 8C, D). Importantly, within the same mice, ILC2 treatment reduced xenoGVHD symptoms similar to what was observed without MV4-11 transfer (FIG. 8E), clearly showing simultaneous protection from pathogeneic T cell responses that underlie GVHD without impairing T cell-mediated GVL effects. Collectively our findings support ILC2s do not limit the anti-leukemic effect of allogeneic T cells despite protecting from GVHD, therefore represent a strong candidate for harnessing ILC2s in cell-based therapies for HSCT.

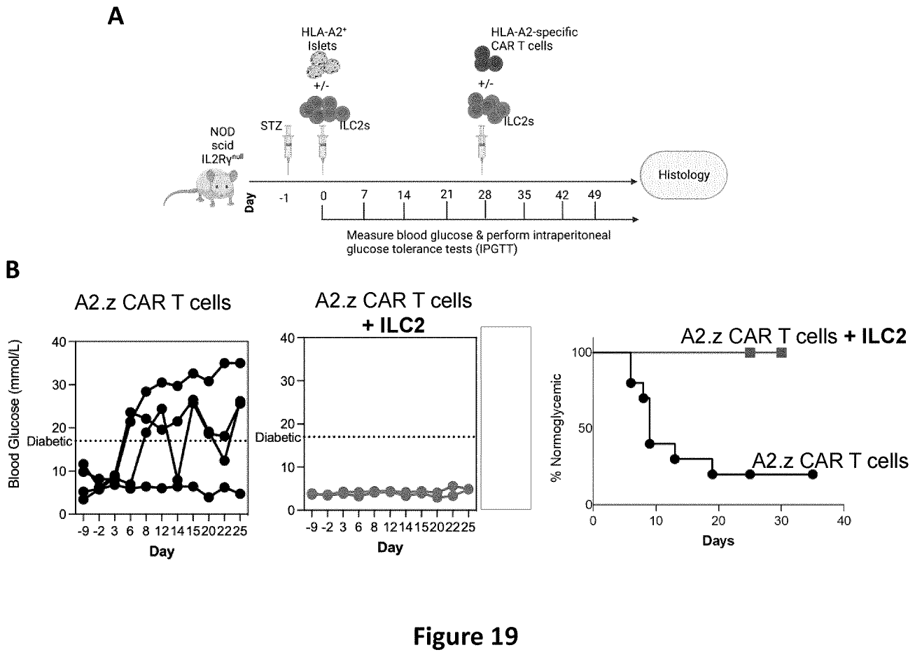

Beyond GVHD, ILC2s would also have cell therapy applications for transplantation and autoimmunity, as adoptive cell therapy of ILC2s inhibits immune-mediated rejection in a humanized model of transplantation (FIG. 19). In this model, non-diabetic 8-14-week-old NOD. Cg-Prkdc^{scid}II2rg^{tm1Wjl}/SzJ (NSG) mice that received 150 mg/kg streptozotocin (STZ) dissolved in 0.1 mol/l citrate buffer, at pH 4.5 intraperitoneally (IP), to induce diabetes and destroy endogenous mouse β-islet cells. 1-2 days later mice which were confirmed to have elevated glycemic levels (>11 mmo/l) were transplanted with human HLA-A+2 cadaveric islets under the left kidney capsule. Each mouse received 1500 IEQ HLA-A2⁺ islets and engraftment was confirmed over a 28 day period. After 28 days, mice which had successful engraftment (determined by consistent blood glucose (BG)<11 mmo/l), received 1.5×10⁶ anti-HLA-A2 CAR T cells (CD4 and CD8 in equal proportions) intravenously (IV) to induce rejection of transplanted islets with or without 1.5×10⁶ expanded ILC2s. The ability of ILC2s to prevent rejection of transplanted islets was assessed by 4 hour fasted blood glucose monitoring and intraperitoneal glucose-tolerance tests (IPGTT), in which mice were then challenged with 200 mg/kg glucose. Mice were deemed diabetic if fasted glucose readings reached >17 mmol/l. All glucose readings were taken on an Accu-Chek Guide glucometer (Roche). As shown in FIG. 19B, mice receiving cell therapy with human ILC2s were protected from T cell mediated destruction of transplanted islets, supporting applications of human ILC2s in inhibiting harmful immune responses in transplantion as well as autoimmunity (in this example type 1 diabetes).

### Discussion

This study demonstrates the cell therapy potential of human IL-10-producing ILC2s to regulate harmful T cell responses, in particular those that drive GVHD pathology. ILC2s therapy inhibited development of GVHD in humanized mouse models without impairing the GVL effects important for success of HSCT. These findings correlate with patient-based studies where high ILC2 proportions are associated with protection from GVHD without increased risk of cancer relapse. CD4⁺ and CD8⁺ T cells within the xenoGVHD model had reduced proliferation, tissue trafficking, intestinal infiltration, as well as decreased proportions of CD4⁺ Th1 and CD8⁺ Tc1 cells across multiple tissue sites with ILC2s therapy. ILC2s expanded using our approach expressed high amounts of IL-10, in line with ILC2₁₀ identity, and were remarkably stable. CD86 and CD49d marked ex vivo expanded IL-10⁺ILC2s, which suppressed IFN-γ expression by CD4⁺ and CD8⁺ T cells through a combination of IL-10 and IL-4. Altogether, our findings support that human ILC2₁₀ limit harmful allogeneic T cell responses and may have applications in adoptive cell therapies aimed at limiting harmful immune responses.

A wide range of prophylactic therapeutics have been explored to treat and prevent aGVHD, including post-transplant cyclophosphamide, calcineurin inhibitors like tacrolimus or cyclosporine and other immunosuppressants like mycophenolate, anti-thymocyte globulin and sirolimus. While these treatments dramatically improved aGVHD outcomes, development of severe aGVHD remains a clinical challenge and there are very limited treatments for those that fail these therapeutics. In a recent multicenter review which included a wide range of different treatment modalities, more than half of HSCT patients required a hospital stay within 100 days of transplant, with the primary reason being development of aGVHD, and severe GVHD (grades III-IV) occurring in 41.9% of patients (42, 43). Patients diagnosed with severe aGVHD were primarily managed with increased steroid doses (51.3% of patients), however, at follow-up, 52.8% of these patients were deceased (42, 43). Therefore, novel therapies are still greatly needed to improve success of HSCT.

Previous studies have reported ILCs are particularly sensitive to pre-transplant conditioning therapies and are depleted from peripheral blood and tissues (15, 38, 39). Unlike NK cells, helper ILCs are slow to reconstitute, further resulting in a reduction in helper-like ILCs after HSCT. Our results add to these prior studies by demonstrating an inverse relationship between ILC2s and CD4⁺ Th1 cells that are associated with GVHD pathology, and demonstrate human ILC2s administered prophylactically or upon GVHD inhibit xenogeneic GVHD. Analysis of ILC2 effects on T cells demonstrated a novel protective mechanism of ILC2s; namely the ability to inhibit allogeneic CD4⁺ Th1 and CD8⁺ Tc1 cells directly through the combination of IL-4 and IL-10. This is a distinct from prior studies of mouse ILC2s, where ILC2s protected via recruitment of immunosuppressive MDSC to the intestine via IL-13 in a major MHC-mismatch BMT model of GVHD, and when mouse ILC2s lost IL-13, T cells were not inhibited as MDSC were not recruited (15, 22). Using this xenogeneic GVHD model, the vast majority of human CD45⁺ cells engrafted are T cells, with limited contribution from myeloid cells, NK cells and B cells. Therefore, while we were able to evaluate effects of ILC2s on T cells, additional protective mechanism may exist that cannot be captured using this xenogeneic mouse model.

CD127⁺CRTh2⁺ILC2s are heterogeneous and their cytokine production is influenced by activating cytokines (16, 17, 19-21, 34). While bulk expanded ILC2s successfully limited development of xenoGVHD, this may be enhanced by isolating only ILC2s that express IL-4 and IL-10. In one of the few studies that considered unique makers of ILC2₁₀, Golebski et al. reported KLRG1⁺ILC2s produced IL-10 along with other signature ILC2 cytokines (21). Here CITE-seq analysis of expanded human IL-10⁺ILC2s revealed limited expression of KLRG1, but instead expressed high levels of CD49d and CD86. Flow cytometry confirmed that ILC2s co-expressing CD49d and CD86 had the greatest expression of IL-10, supporting these markers could be used to isolate or improve purity of ILC2₁₀ in expansion protocols.

Tregs can dramatically reduce aGVHD severity in preclinical mouse models and clinical trials have noted evidence of Tregs limiting allo-immune responses. However, use of polyclonal Tregs has not achieved significant response rates, and the majority of patients still develop GVHD or require global immunosuppression (refs). Further, Tregs therapies have been limited by issues including impeding the GVL effects, difficulties with Treg stability and purity in expansions, and limited effectiveness when using polyclonal Tregs (40). Additionally, Tregs are typically sourced from the transplant donor or recipient, necessitating time sensitive isolation and expansion (41). We show here that expanded human ILC2s that are allogeneic to PBMCs suppress xenoGVHD severity while preserving the GVL effect, supporting 3^{rd} party donors could be used as a source of “off-the-shelf” ILC2s for cell therapy. Further, studies in mice have shown that ILC2-derived amphiregulin protects from intestinal tissue damage during GVHD, an additional reparative benefit compared to Tregs (15, 22). As expanded human IL-10⁺ILC2s also express high levels of amphiregulin, harnessing dual immunoregulatory and reparative functions may represent an additional advantage of ILC2₁₀ for GVHD and other indications.

ILC2s display significant heterogeneity and can adopt proinflammatory or tissue protective functions depending on cytokines and microenvironment factors. ILC2₁₀ have been shown to inhibit immune responses in diverse contexts, including inflammation in the lungs, intestines, and neurological system. In humans, individuals with grass-pollen allergies have ILC2s with reduced capacity to produce IL-10 following stimulation, and patients who respond to allergy immunotherapy have increased proportions of IL-10⁺ILC2s (21). In the context of transplantation, Huang et al. demonstrated mouse ILC2₁₀ could prolong islet allograft survival by suppressing T cell attack of transplanted islets (34). While these growing reports demonstrate important functions for ILC2₁₀ in regulating immunity or instead contributing to proinflammatory responses, we have lacked markers that accurately identify ILC2₁₀ from conventional or inflammatory ILC2s. In one of the few studies that attempted to identify unique markers of ILC2₁₀, Golebski et al. reported KLRG1⁺ILC2s produced IL-10 along with other signature ILC2 cytokines (21). However, KLRG1 has also been associated with inflammatory ILC2s, and may instead mark activated or memory like ILC2 populations, not specifically ILC2₁₀(57-60). CITE-seq analysis of activated and expanded human ILC2₁₀ revealed limited expression of KLRG1, but instead high levels of CD49d and CD86. Flow cytometry confirmed that ILC2s co-expressing CD49d and CD86 had the greatest expression of IL-10. To assess the broader utility of these markers, we assessed whether these markers correlated with ILC2₁₀ observed from grass-pollen allergy patients that responded to allergen immunotherapy in Golebski et al. ITGA4 (CD49d) and CD86 increased following allergy immunotherapy, in addition to KLRG1 previously reported. Ex vivo expanded ILC2₁₀ from peripheral blood did not express KLRG1, however, which may indicate KLRG1 is downregulated in ex vivo cultures, or that KLRG1 is differentially expressed by ILC2₁₀ in different tissues or contexts. The combination of CD49d and CD86 however distinguished IL-10⁺ILC2s from conventional ILC2s in both studies, demonstrating their utility in tracking ILC2₁₀ responses in human studies.

ILC2₁₀ have been shown to limit inflammation the diverse contexts. Murine lung ILC2s that limited inflammation following IL-2 administration showed a dramatic induction in IL-10, and intestinal mouse ILC2s were primary producers of IL-10 at steady state and during disease (17). Individuals with grass-pollen allergies have ILC2s with reduced capacity to produce IL-10 following stimulation, and patients who respond to allergy immunotherapy have increased proportions of IL-10⁺ILC2s (21). In the context of transplantation, Huang et al. demonstrated murine IL-10⁻producing ILC2s could prolong islet allograft survival by limiting allogeneic T cell attack of transplanted islets (34). These growing reports demonstrate important functions for ILC2₁₀ in regulating immunity, and taken with our study provide a strong rational for ILC2₁₀-based cell therapies in other contexts, including autoimmunity and transplantation.

To this end we also demonstrate human ILC2s could prevent T cell mediated attack in a mouse model of transplantation (FIG. 19). Islet transplant is a potentially curative therapy for individuals with T1D as it restores insulin-producing β-cells and has the capacity to normalize glycemic control. However, individuals receiving islet transplant therapies require life-long harmful immunosuppressive regimes in order to inhibit reoccurring autoimmunity and allogeneic immune reactions. While other tolerance-promoting adoptive cellular therapies are being explored, ILC2s represent a promising avenue due to their capacity to suppress T cells in an allogeneic context. Here, we harnessed human ILC2s to prevent rejection of transplanted islets, supporting potential applications both for transplantation and autoimmunity.

### EXAMPLE 2

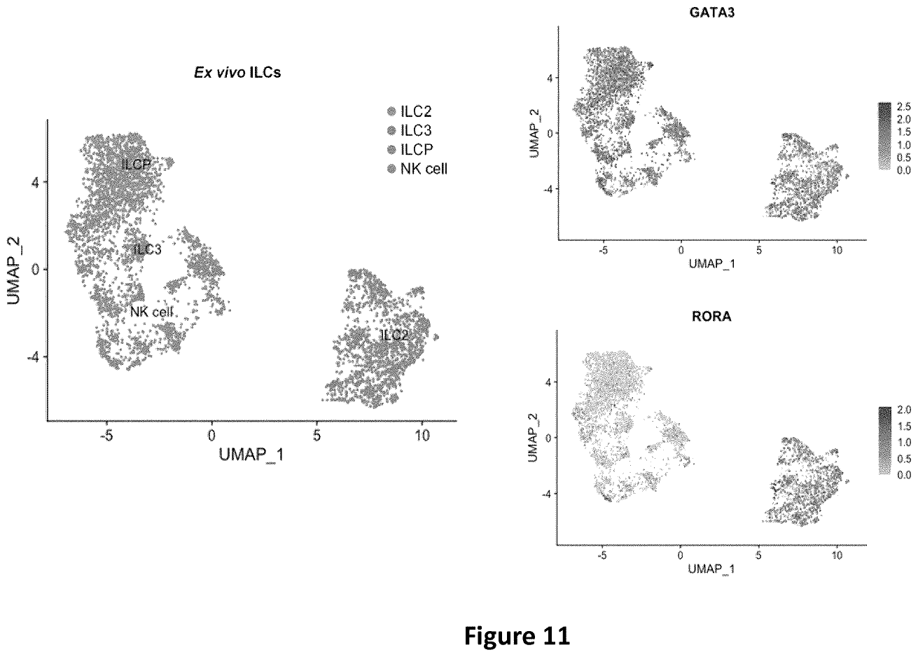

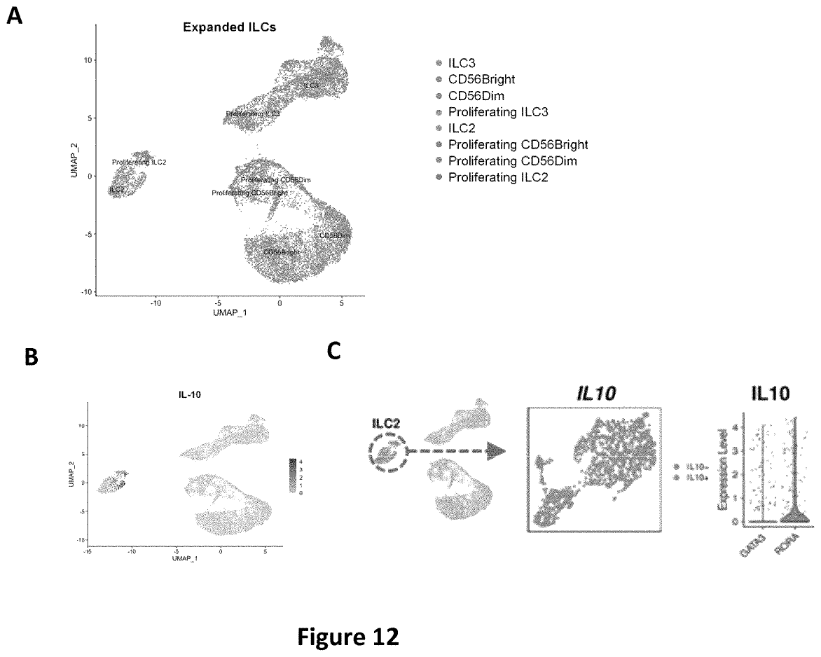

Applicant further explored a subpopulation of ILC2s that produces IL-10 and its ability to limit T cell responses in islet transplant, helminth expulsion and allergic rhinitis. Features that differentiate IL-10⁺ ILC2s from ILC2s that do not produce IL-10 are largely unknown, and currently transcription factors which promote IL-10 production by ILC2s have not been defined. To explore IL-10⁺ ILC2 biology and applications in tolerogenic cell therapies, we developed methods to isolate and expand human IL-10⁺ ILC2s and identified markers selectively expressed by IL-10⁺ ILC2s. Using CITEseq, we assessed transcription factor expression by human ILC2s, specifically IL-10⁺ ILC2s. Ex vivo ILC2s from blood selectively expressed RAR Related Orphan Receptor A (RORA) but GATA-3 was expressed at the transcript level by all human ILCs (FIG. 11). Upon activation of ILC2s with cytokines used to expand human ILC2s described above, IL-10 expression was detected. When expression of IL-10 was assessed in activated human ILC2s, IL-10 expression was selectively detected in ILC2s that expressed RORA but not GATA-3 (FIG. 12B-C).

### Materials and Methods

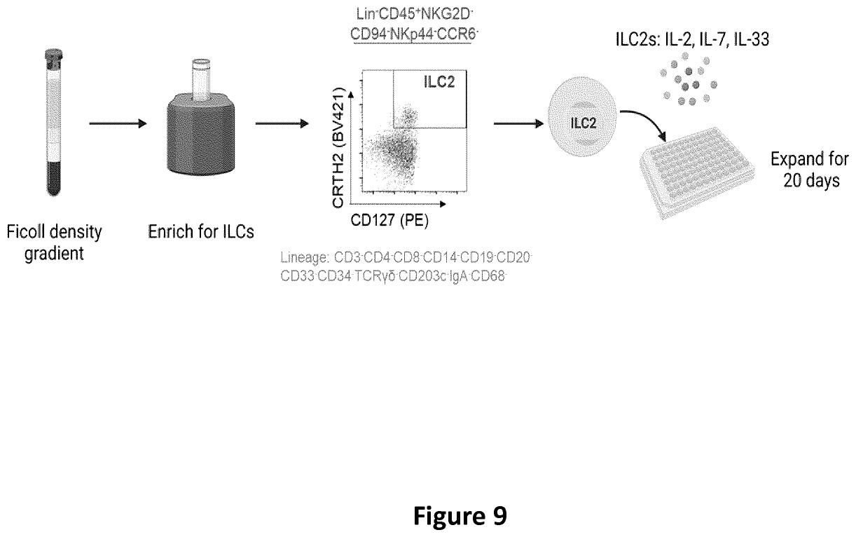

FIG. 9 shows a general schematic of isolation and expansion of human ILC2s. PBMCs were isolated using a Ficoll density gradient and are enriched for ILCs using a combination of lineage depletion and purification by FACS sorting. ILC2s were sorted as Lin⁻, CD45⁺, NKG2D, CD94⁻, NKp44⁻, CCR6⁻, CD127⁺, and CRTH2⁺. ILC2s were cultured in their activating cytokines for 20 days.

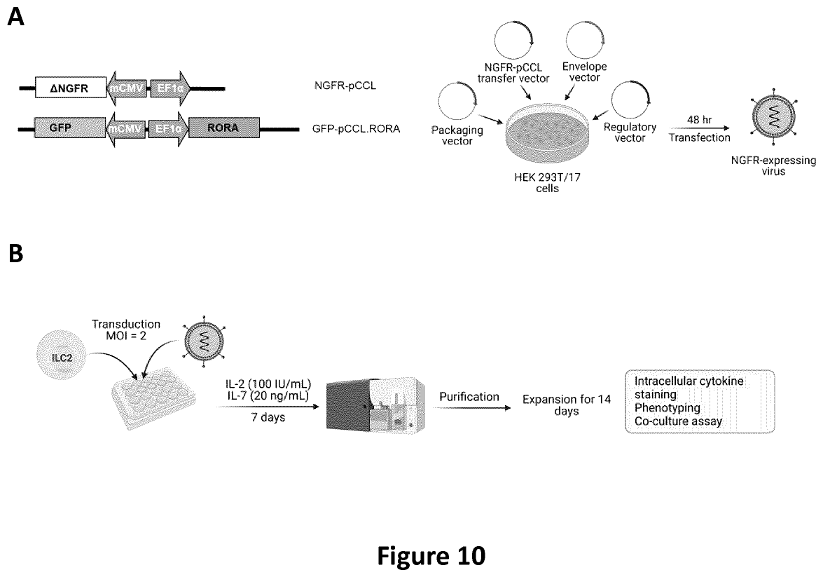

Referring to FIG. 10A, the bi-directional lentiviral vectors expressing the non-signaling nerve growth factor receptor (NGFR) or GFP marker gene and ILC-associated transcription factors RORA used for these studies. Lentivirus was produced by transient four-plasmid 48-hour transfection of HEK 293T cells.

FIG. 10B shows the general scheme for transduction of human canonical ILC2s.

### Results and Discussion

Referring to FIG. 11, there is shown RORA expression distinguishes ex vivo human ILC2s. UMAP plots of ex vivo ILC subsets after cellular indexing of transcriptomes and epitopes sequencing (CITEseq), and corresponding RNA expression of GATA3 and RORA.

Ex vivo expanded and activated IL-10 producing ILC2s express RORA, and little to no GATA-3, whereas ILC2s that lack IL-10 production express GATA3 (FIG. 12B-C). FIG. 12A shows UMAP plots of expanded ILC subsets after CITE-seq. ILC subsets were isolated using FACS, expanded and separately stained with an antibody cocktail. Each cell population was stained with a unique hashtag antibody to allow identification post sequencing. FIG. 12B shows UMAP plots showing RNA level expression of IL-10, on expanded ILC subsets. FIG. 12C shows IL-10 expressing cells selectively express RORA.

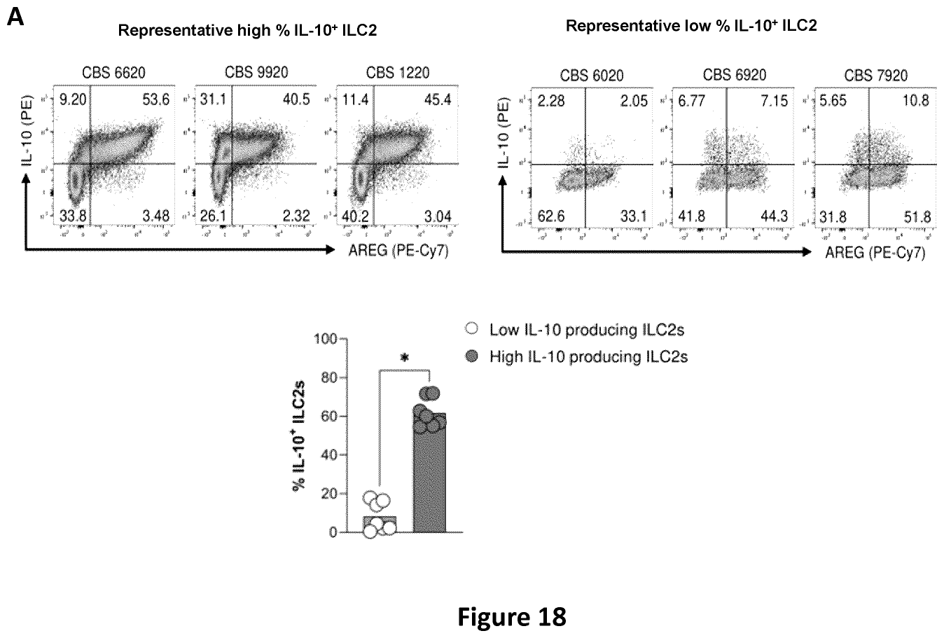

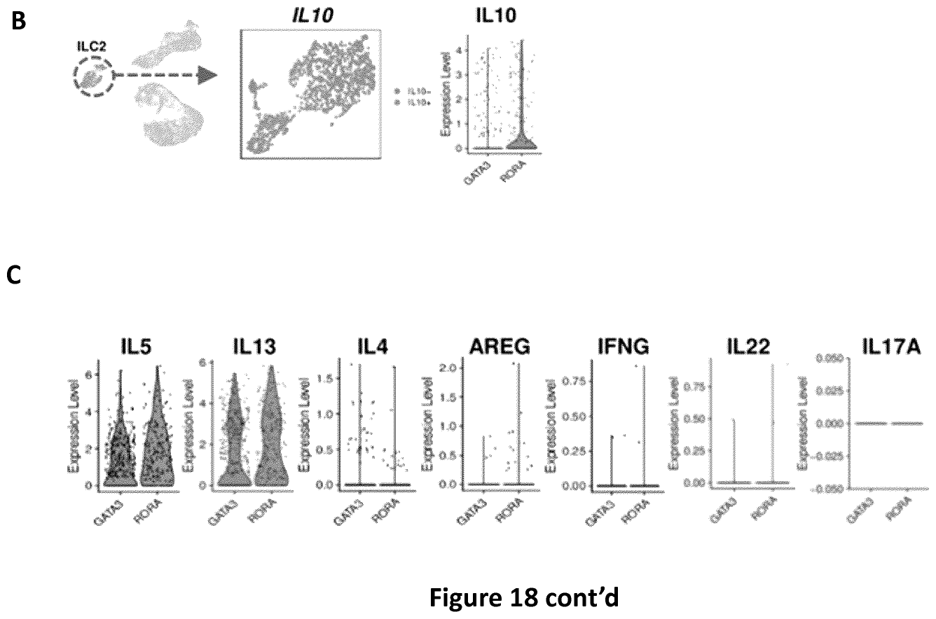

FIG. 18 demonstrates variable proportions of IL-10-expressing ILC2s in ILC2 preparations from different donors. Representative flow cytometry plots after intracellular cytokine staining (ICS) on expanded ILC2s, showing three high IL-10 producing donors, and three low IL-10 producing donors, as well as summary plots of IL-10 expression in ILC2 cell preparations from 12 independent PBMC donors (FIG. 18A). CITEseq analysis of IL-10 expressing ILC2s demonstrates IL-10-expressing cells express the transcription factor RORA (FIG. 18B) but not GATA3. However no difference in expression of other ILC2-associated molecules including IL-4, IL-5, IL-13 and amphiregulin (AREG), or upregulation of ILC1 or ILC3 associated cytokines (IFN-g and IL22/IL-17A respectively) was observed in RORA versus GATA3 expressing ILC2s.

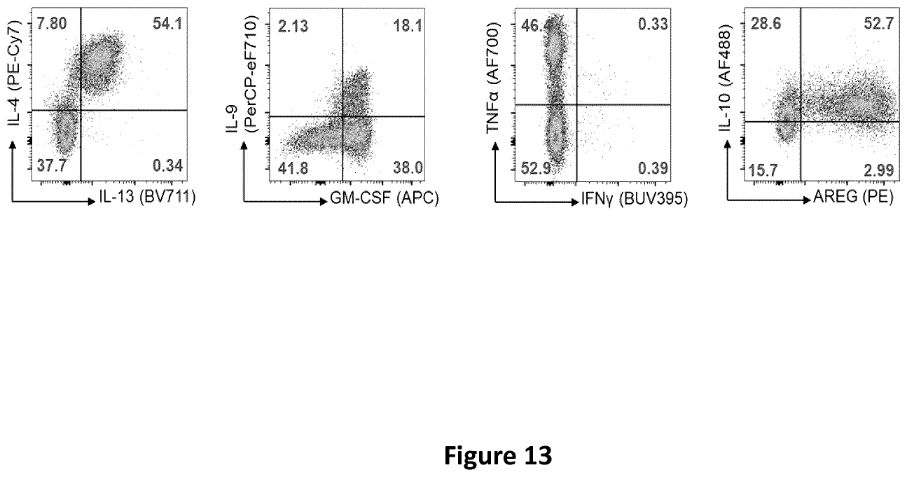

Human ILC2s expanded from healthy blood PBMCs in vitro maintain expression of signature cytokines and produce immunoregulatory cytokine IL-10. FIG. 13B shows representative flow plots of intracellular cytokine staining in expanded human ILC2s, after stimulation with PMA/lonomycin for 6 hours. Protein inhibitors, Brefeldin A and Monensin were added after 2 hours of stimulation.

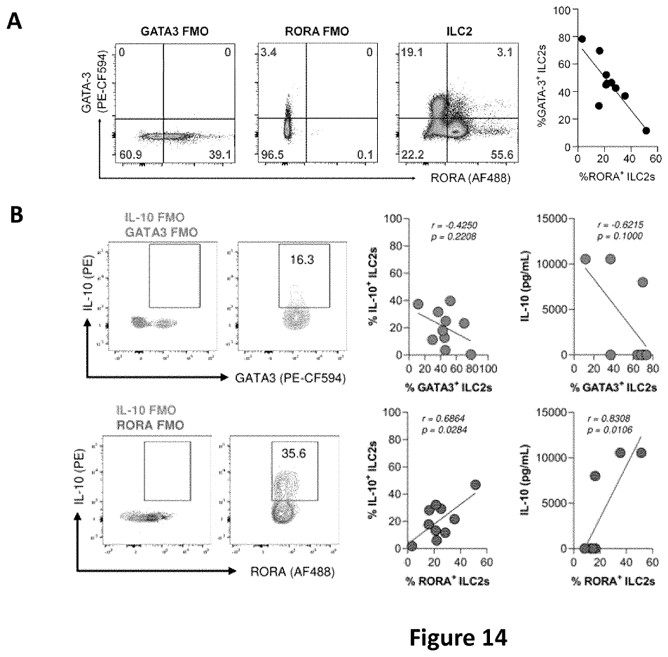

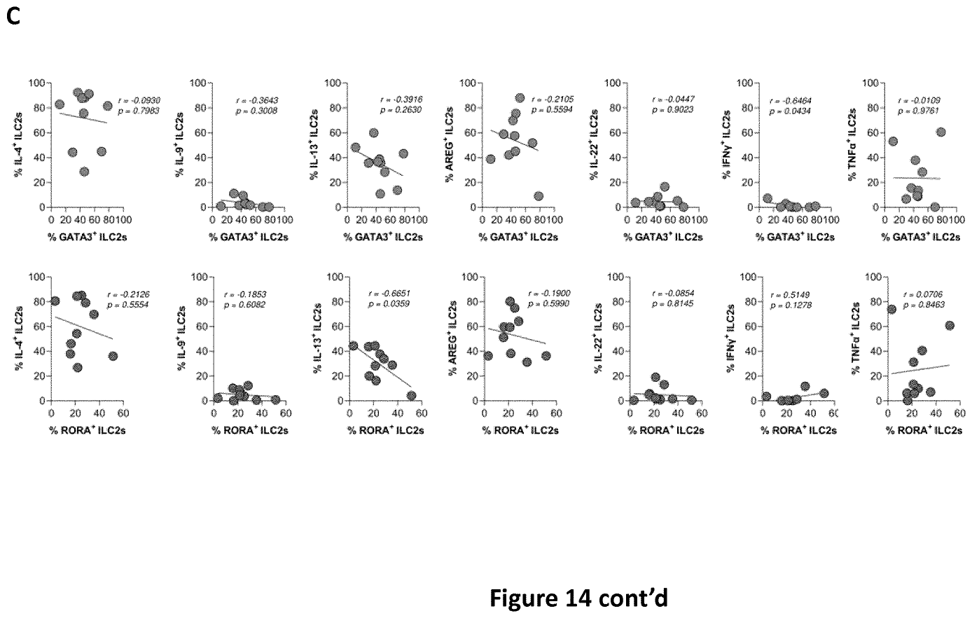

RORA expression positively correlates with IL-10 production by ILC2s. FIG. 14A shows representative flow plots of transcription factor staining of expanded human ILC2s, demonstrating that following cytokine stimulation, human ILC2s differentially express GATA-3 and RORA at the protein level. FIG. 14B shows representative flow plots of intracellular cytokine staining of human GATA3⁺ and RORA⁺ ILC2s. FIG. 14B demonstrates IL-10 expression correlates with RORA-expression but GATA-3 expression is inversely correlated. RORA-expression also inversely correlated with IL-13 expression.

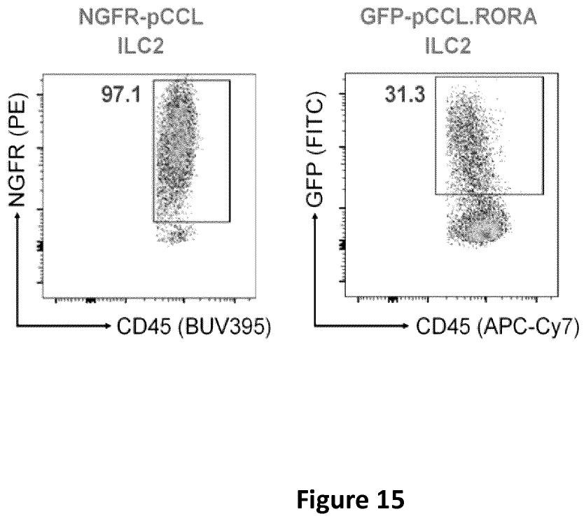

To determine whether RORA-expression promotes IL-10 production by human ILC2s, human ILC2s were transduced with NGFR-pCCL and GFP-pCCL. RORA lentiviral vectors, at a multiplicity of infection of 2. Transduction efficiency was measured 7 days post-transduction by assessing NGFR or GFP expression (FIG. 15C).

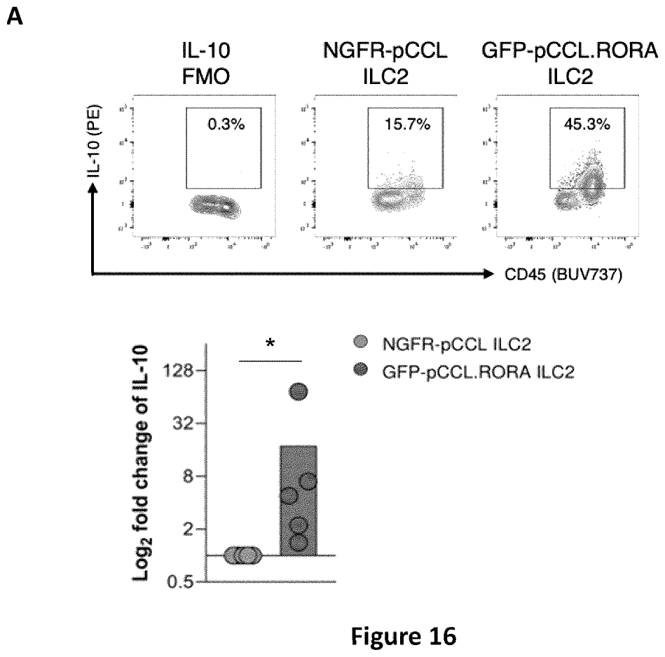

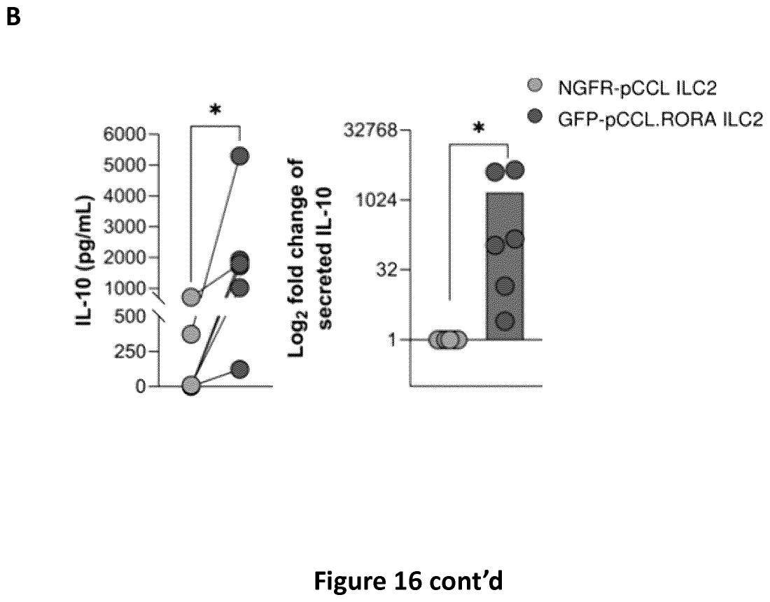

Overexpression of RORA induces IL-10 production in ILC2s. Cytokine expression by GFP-pCCL. RORA ILC2s was quantified by intracellular cytokine production by flow cytometry (FIG. 16B) and cytometric bead array (FIG. 16C). NGFR-pCCL control transduced ILC2s were tested in parallel to compare changes in cytokine production. RORA transduction upregulated IL-10 production by human ILC2s (FIG. 16B,C).

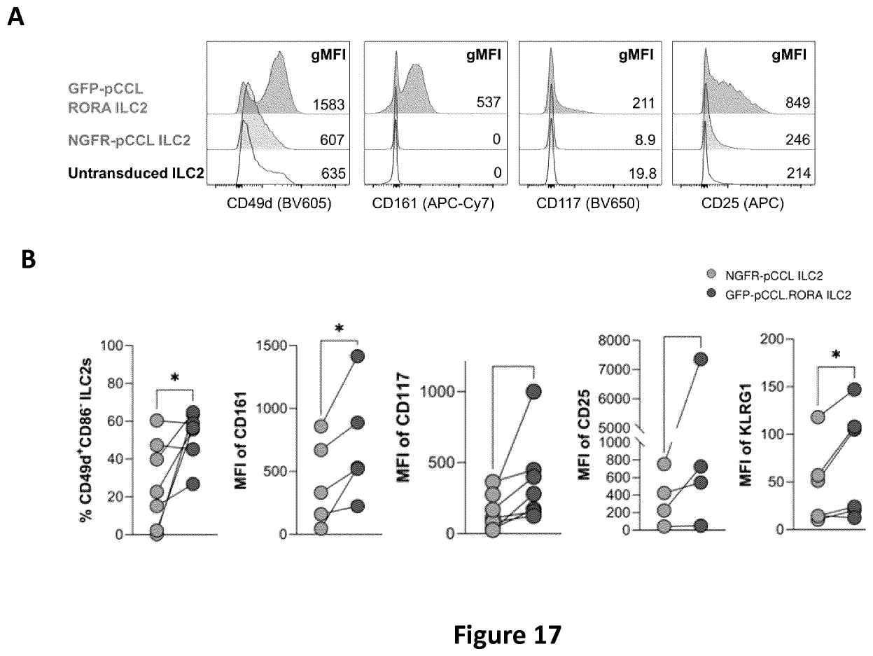

In addition to upregulating IL-10 expression, overexpression of RORA upregulates surface expression of CD49d, a marker defining IL-10 producing ILC2s (FIG. 17A-B), as well as CD161, CD117, CD25 and KLRG1. Surface marker expression by GFP-pCCL. RORA ILC2s were quantified by flow cytometry. NGFR-pCCL control transduced ILC2s were tested in parallel to compare changes in surface marker expression.

In summary, we show that ex vivo activated human ILC2s that produce IL-10 express RORA, whereas ILC2s that lack IL-10 production express GATA3. At the protein level, RORA expression positively correlates with IL-10 production. Using a lentiviral gene transfer system, we demonstrate enforced RORA expression in human ILC2s promotes IL-10 production in ILC2s, supporting that enforced expression of RORA supports IL-10-producing ILC2s. Therefore RORA-overexpression represents a method to induce or engineer IL-10-producing ILC2s, with applications for cell therapies in GVHD, autoimmunity, transplantation and inflammation.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Garnett C, Apperley J F, Pavlu J. Treatment and management of graft-versus-host disease: Improving response and survival. Ther Adv Hematol 2013;4(6):366-378.
2. Burman A C et al. IFNγ differentially controls the development of idiopathic pneumonia syndrome and GVHD of the gastrointestinal tract. Blood 2007;110(3):1064-1072.
3. Guy-Grand D, Vassalli P. Gut injury in mouse graft-versus-host reaction. Study of its occurrence and mechanisms. Journal of Clinical Investigation 1986;77(5):1584-1595.
4. Gutiérrez-Hoya A et al. Role of CD8 Regulatory T Cells versus Tc1 and Tc17 Cells in the Development of Human Graft-versus-Host Disease. J Immunol Res 2017; 2017. doi: 10.1155/2017/1236219
5. Szebeni J, Wang M G, Pearson D A, Szot G L, Sykes M. IL-2 inhibits early increases in serum gamma interferon levels associated with graft-versus-host-disease.. Transplantation 1994;58(12):1385-93.
6. Xhaard A et al. Steroid-Refractory Acute GVHD: Lack of Long-Term Improved Survival Using New Generation Anticytokine Treatment. Biology of Blood and Marrow Transplantation 2012;18(3):406-413.
7. Cheung T S et al. Mesenchymal Stromal Cells for Graft Versus Host Disease: Mechanism-Based Biomarkers. Front Immunol 2020; 11. doi:10.3389/fimmu.2020.01338
8. Elias S, Rudensky A Y. Therapeutic use of regulatory T cells for graft-versus-host disease. Br J Haematol 2019;187(1):25-38.
9. Koehn B H, Blazar B R. Role of myeloid-derived suppressor cells in allogeneic hematopoietic cell transplantation.. J Leukoc Biol 2017;102(2):335-341.
10. Simonetta F, Alvarez M, Negrin R S. Natural killer cells in graft-versus-host-disease after allogeneic hematopoietic cell transplantation. Front Immunol 2017;8(APR). doi:10.3389/fimmu.2017.00465
11. Minculescu L et al. Early Natural Killer Cell Reconstitution Predicts Overall Survival in T Cell-Replete Allogeneic Hematopoietic Stem Cell Transplantation. Biology of Blood and Marrow Transplantation 2016;22(12):2187-2193.
12. Marius Munneke J et al. Activated innate lymphoid cells are associated with a reduced susceptibility to graft-versus-host disease. Blood 2014;124(5):812-821.
13. Hanash A M et al. Interleukin-22 Protects Intestinal Stem Cells from Immune-Mediated Tissue Damage and Regulates Sensitivity to Graft versus Host Disease. Immunity 2012;37(2):339-350.
14. Hazenberg M D et al. Human ectoenzyme-expressing ILC3:Immunosuppressive innate cells that are depleted in graft-versus-host disease. Blood Adv 2019;3(22):3650-3660.
15. Bruce D W et al. Type 2 innate lymphoid cells treat and prevent acute gastrointestinal graft-versus-host disease. Journal of Clinical Investigation 2017;127(5):1813-1825.
16. Jegatheeswaran S, Mathews J A, Crome S Q. Searching for the Elusive Regulatory Innate Lymphoid Cell. The Journal of Immunology 2021;207(8):1949-1957.
17. Seehus C R et al. Alternative activation generates IL-10 producing type 2 innate lymphoid cells. Nat Commun 2017;8(1). doi:10.1038/s41467-017-02023-z
18. Howard E et al. IL-10 production by ILC2s requires Blimp-1 and cMaf, modulates cellular metabolism, and ameliorates airway hyperreactivity. Journal of Allergy and Clinical Immunology 2021;147(4):1281-1295.e5. ⁻19. Nc D et al. Meningeal type-2 innate lymphoid cells emerge as novel regulators of microglial activation and blood-brain barrier stability: a central role for IL-10 [Internet]. 2019:
20. Bando J K et al. ILC2s are the predominant source of intestinal ILC-derived IL-10. Journal of Experimental Medicine 2020;217(2). doi:10.1084/jem.20191520
21. Golebski K et al. Induction of IL-10-producing type 2 innate lymphoid cells by allergen immunotherapy is associated with clinical response. Immunity 2021;54(2):291-307.e7.
22. Bruce D W et al. Third-party type 2 innate lymphoid cells prevent and treat GI tract GvHD.. Blood Adv 2021;5(22):4578-4589.
23. Ehx G et al. Xenogeneic Graft-Versus-Host Disease in Humanized NSG and NSG-HLA-A2/HHD Mice. Front Immunol 2018;9. doi:10.3389/fimmu.2018.01943
24. Croudace J E et al. Chemokine-mediated tissue recruitment of CXCR3 CD4 T cells plays a major role in the pathogenesis of chronic GVHD [Internet][published online ahead of print:2012]; doi:10.1182/blood
25. Choi J et al. IFNR signaling mediates alloreactive T-cell trafficking and GVHD [Internet]. 2012:
26. Crome S Q, Wang A Y, Kang C Y, Levings M K. The role of retinoic acid-related orphan receptor variant 2 and IL-17 in the development and function of human CD4⁺ T cells. Eur J Immunol 2009;39(6):1480-1493.
27. Crome S Q et al. Inflammatory Effects of Ex Vivo Human Th17 Cells Are Suppressed by Regulatory T Cells. The Journal of Immunology 2010;185(6):3199-3208.
28. Acosta-Rodriguez E v. et al. Surface phenotype and antigenic specificity of human interleukin 17-producing T helper memory cells. Nat Immunol 2007;8(6):639-646.
29. Cosmi L et al. CRTH2 is the most reliable marker for the detection of circulating human type 2 Th and type 2 T cytotoxic cells in health and disease. 2000:
30. Chen P P et al. Alloantigen-specific type 1 regulatory T cells suppress through CTLA-4 and PD-1 pathways and persist long-term in patients. Sci Trans Med 2021;13(617). doi:10.1126/scitranslmed.abf5264
31. Gagliani N et al. Coexpression of CD49b and LAG-3 identifies human and mouse T regulatory type 1 cells. Nat Med 2013;19(6):739-746.
32. Ohta A et al. A2A Adenosine Receptor May Allow Expansion of T Cells Lacking Effector Functions in Extracellular Adenosine-Rich Microenvironments. The Journal of Immunology 2009;183(9):5487-5493.
33. Long A et al. Type 2 Innate Lymphoid Cells Impede IL-33-Mediated Tumor Suppression. The Journal of Immunology 2018;201(11):3456-3464.
34. Huang Q et al. IL-10 producing type 2 innate lymphoid cells prolong islet allograft survival. EMBO Mol Med 2020;12(11). doi:10.15252/emmm.202012305
35. Yang L et al. Interleukin-13 interferes with activation-induced t-cell apoptosis by repressing p53 expression [Internet]2015;13. doi:10.1038/cmi.2015.50; published
36. Rauber S et al. Resolution of inflammation by interleukin-9-producing type 2 innate lymphoid cells. Nat Med 2017;23(8):938-944.
37. Murphy K M, Reiner S L. The lineage decisions of helper T cells. Nat Rev Immunol 2002;2(12):933-944.
38. Piperoglou C et al. Innate lymphoid cell recovery and occurrence of GvHD after hematopoietic stem cell transplantation [Internet]. J Leukoc Biol 2022;111(1):161-172.
39. Kroeze A et al. Presence of innate lymphoid cells in allogeneic hematopoietic grafts correlates with reduced graft-versus-host disease. Cytotherapy [published online ahead of print:2022]; doi:10.1016/j.jcyt.2021.10.011
40. Hefazi M, Bolivar-Wagers S, Blazar B R. Regulatory T Cell Therapy of Graft-versus-Host Disease: Advances and Challenges.. Int J Mol Sci 2021;22(18). doi:10.3390/ijms22189676
41. McCallion O, Bilici M, Hester J, Issa F. Regulatory T-cell therapy approaches. Clin Exp Immunol [published online ahead of print: Aug. 12, 2022]; doi:10.1093/cei/uxac078
42. Holtan S G et al. Disease progression, treatments, hospitalization, and clinical outcomes in acute GVHD: a multicenter chart review. Bone Marrow Transplant 2022;57(10):1581-1585.
43. Holtan S G et al. Disease progression, hospital readmissions, and clinical outcomes for patients with steroid-refractory acute graft-versus-host disease: A multicenter, retrospective study. Bone Marrow Transplant 2022;57(9):1399-1404.
44. Dawson N A J et al. Systematic testing and specificity mapping of alloantigen-specific chimeric antigen receptors in regulatory T cells. JCI Insight 2019;4(6). doi:10.1172/jci.insight.123672
45. Liu X, Quan N. Immune Cell Isolation from Mouse Femur Bone Marrow. Bio Protoc 2015;
46. Hao Y et al. Integrated analysis of multimodal single-cell data. Cell 2021;184(13):3573-3587.e29.
47. McInnes L, Healy J, Saul N, Großberger L. UMAP: Uniform Manifold Approximation and Projection. J Open Source Softw 2018;3(29):861.
48. Blighe K, Rana S, Lewis M. EnhancedVolcano:publication-ready volcano plots with enhanced colouring and labeling. Bioconductor version: Release (3.11), 2020.
49. Vivier, E., et al. 2018. Innate lymphoid cells: 10 years on. Cell 174:1054-1066.
50. Murphy J M, Ngai L Mortha M and S Q Crome. Tissue-Dependent Adaptations and Functions of Innate Lymphoid Cells. Front. Immunol., 10 Mar. 2022,
51. Jegatheeswaran S, Mathews J A, Crome S Q. “Searching for the elusive regulatory innate lymphoid cell”. Journal of Immunology. 2021. 207(8):1949-1957
52. S Q Crome, P S Ohashi. Immunoregulatory functions of innate lymphoid cells Journal for Immuno Therapy of Cancer 6 (1), 1-4
53. Rauber, S. et al. Resolution of inflammation by interleukin-9-producing type 2 innate lymphoid cells. Nat. Med. 23, 938-944 (2017).
54. Bruce, D. W. et al. Type 2 innate lymphoid cells treat and prevent acute gastrointestinal graft-versus-host disease. J. Clin. Invest. 127, 1813-1825 (2017).
55. Cao Q, et al. Potentiating Tissue-Resident Type 2 Innate Lymphoid Cells by IL-33 to Prevent Renal Ischemia-Reperfusion Injury. J Am Soc Nephrol. 2018;29(3):961-976.
56. Huang, Q., X. Ma, Y. Wang, Z. Niu, R. Wang, F. Yang, M. Wu, G. Liang, P. Rong, H. Wang, D. C. H. Harris, W. Wang, and Q. Cao. 2020. IL-10 producing type 2 innate lymphoid cells prolong islet allograft survival. EMBO Mol. Med. 12:e12305:
57. Blanquart E, et al. Targeting androgen signaling in ILC2s protects from IL-33-driven lung inflammation, independently of KLRG1. Journal of Allergy and Clinical Immunology. 2022;149(1):237-251.e12.
58. Taylor S, et al. PD-1 regulates KLRG 1⁺ group 2 innate lymphoid cells. Journal of Experimental Medicine. 2017;214(6):1663-1678.
59. Flamar A-L, et al. Interleukin-33 Induces the Enzyme Tryptophan Hydroxylase 1 to Promote Inflammatory Group 2 Innate Lymphoid Cell-Mediated Immunity. Immunity. 2020;52(4):606-619.e6.
60. Huang Y, et al. IL-25-responsive, lineage-negative KLRG1hi cells are multipotential ‘inflammatory’ type 2 innate lymphoid cells. Nat Immunol. 2015;16(2):161-169.

1. A method of isolating or enriching a population of cells for IL-10 producing group 2 innate lymphoid cells, the method comprising:
providing the population of cells which comprises ILC2s;
selecting for cells expressing at least one of three markers: CD86, CD49d and retinoic acid related orphan alpha (RORA) and/or deselecting for cells that express GATA3.

2. The method of claim 1, wherein cells are selected that express CD86.

3. The method of claim 1, wherein cells are selected that express CD49d.

4. The method of claim 1, wherein cells are selected that express RORA.

5. The method of claim 1, wherein cells are selected that express both CD86 and CD49d.

6. The method of claim 1, wherein cells are selected that express all of CD86, CD49d and RORA.

7. The method of claim 1, further comprising deselecting for cells that express GATA3.

8. The method of claim wherein the population of cells is comprised in a sample from a subject.

9. The method of claim 8, wherein the sample is blood, bone marrow, tissue or a lymphoid organ.

10. The method of claim 1, for isolating IL-10 producing group 2 innate lymphoid cells.

11. The method of claim 1, wherein the enriched population of cells is intended for use in the ameliorating, treating or preventing of graft-versus-host disease, transplant rejection, an inflammatory condition, an autoimmune disorder, allergy, asthma, promoting transplant graft function and/or preventing or limiting tissue damage in a human subject in need thereof.

12. The method of claim further comprising administering to a subject in need thereof, a therapeutically effective amount of the population of cells enriched for IL-10 producing group 2 innate lymphoid cells, for ameliorating, treating or preventing graft-versus-host disease, transplant rejection, an inflammatory condition, an autoimmune disorder, allergy, asthma, promoting transplant graft function and/or preventing or limiting tissue damage in the subject.

13. The method of claim 12, wherein the subject has undergone or will undergo a cell transplantation procedure.

14. The method of claim 13, wherein the cell transplantation is stem cell transplantation, the latter preferably being hematopoietic stem cell transplantation.

15. The method of claim 12, wherein the subject has undergone or will undergo a bone marrow transplantation procedure.

16. The method of claim 12, wherein the subject has undergone or will undergo a solid organ transplantation procedure.

17. The method of claim 12, wherein the subject has undergone or will undergo a stem cell derived transplantation procedure of tissues, cells or organs.

18. A population of cells enriched for IL-10 producing group 2 innate lymphoid cells prepared by the method of claim 1.

19. (canceled)

20. A kit for enriching a population of cells for IL-10 producing group 2 innate lymphoid cells, the kit comprising reagents for detecting cells expressing at least one of four markers, CD86, CD49d, RORA and GATA3 along with instructions for use.

21. (canceled)

22. (canceled)

23. (canceled)

24. (canceled)

25. (canceled)

26. (canceled)

27. A method of generating or enriching for a population of cells for IL-10 producing group 2 innate lymphoid cells, the method comprising:
providing the population of cells which comprises ILC2s;
inducing overexpression of RORA in the population of cells.
